(19) United States
(12) Patent Application Publication
(10) Pub. No.: US 2007/0275944 A1
(43) Pub. Date: Nov. 29, 2007

(54) Antioxidants and methods of their use

(75) Inventor: Martyn A. Sharpe, East Lansing, MI (US)

Correspondence Address: MEDLEN & CARROLL, LLP, 101 HOWARD STREET, SUITE 350, SAN FRANCISCO, CA, 94105, US

(73) Assignee: The Board of Trustees Operating Michigan State University

(21) Appl. No.: 11/732,754

(22) Filed: Apr. 4, 2007

Related U.S. Application Data
(60) Provisional application No. 60/789,307, filed on Apr. 5, 2006.

(51) Int. Cl. A61K 31/54; C07D 293/12; A61K 31/421; A61K 31/496; A61K 31/513; C07D 421/02; C07D 403/02

(52) U.S. Cl. 514/183; 544/1; 514/269; 544/260; 514/253.01; 544/314

(57) ABSTRACT

The invention relates to compounds and compositions to treat some neurodegenerative diseases. In some embodiments, the invention relates to an antioxidant comprising a selenium atom and nitroxide group. In further embodiments, the antioxidant comprises peroxidase and superoxide dismutase activity. In some embodiments, the antioxidants are effective in treating neurodegenerative diseases including, but not limited to, Alzheimer's disease, Parkinson's disease, or multiple sclerosis. In additional embodiments, the invention relates to using compounds disclosed herein as free radical electromagnetic imaging agents.

### FIELD OF INVENTION

The invention relates to compounds and compositions to treat some neurodegenerative diseases. In some embodiments, the invention relates to an antioxidant comprising a selenium atom and nitroxide group. In further embodiments, the antioxidant comprises peroxidase and superoxide dismutase activity. In some embodiments, the antioxidants are effective in treating neurodegenerative diseases including, but not limited to, Alzheimer's disease, Parkinson's disease, or multiple sclerosis. In additional embodiments, the invention relates to using compounds disclosed herein as free radical electromagnetic imaging agents.

### BACKGROUND

Reactive oxygen species and reactive nitrogen species occur during natural metabolism, and the immune system can create them on purpose to rid itself of foreign material such as bacteria and virus. The human body can handle certain levels of these species, but excessive levels cause severe damage to cells and tissues. Compounds that act as antioxidants can reduce the levels of these species. For example, vitamin C, vitamin E and selenium are antioxidants recommended in a daily diet by the FDA. However, there is a continued need to identify antioxidants for improved health.

The pathogenesis of several diseases, including cancer and neurodegenerative diseases such as Parkinson's disease, involve the generation of reactive oxygen and nitrogen species associated with mitochondrial dysfunction. However, current therapeutic antioxidants do not have satisfactory pharmacological profiles to effectively manage these diseases. Thus, there is a need to identify compounds that can be used to improve health and manage, prevent, and treat diseases associated with excessive formation reactive oxygen and nitrogen species that do not result in adverse effects in a subject.

### SUMMARY OF INVENTION

The invention relates to compounds and compositions to treat some neurodegenerative diseases. In some embodiments, the invention relates to an antioxidant comprising a selenium atom and nitroxide group. In further embodiments, the antioxidant comprises peroxidase and superoxide dismutase activity. In some embodiments, the antioxidants are effective in treating neurodegenerative diseases including, but not limited to, Alzheimer's disease, Parkinson's disease, or multiple sclerosis. In additional embodiments, the invention relates to using compounds disclosed herein as free radical electromagnetic imaging agents.

In additional embodiments, the invention relates to a nutritional supplement or therapeutic compositions that contain compounds disclosed herein and methods of administering said compositions to a subject.

In some embodiments, the invention relates to a method, comprising modifying an antioxidant by introducing a tetrahydropyridine moiety. In additional embodiments, the invention relates to a method for treating a neurodegenerative disease, comprising: a) providing; i) a patient, wherein said patient has a neurodegenerative disease characterized by high oxidative stress; and ii) a composition comprising a tetrahydropyridine moiety conjugated to an antioxidant compound; b) administering said composition to said patient under conditions such that said oxidative stress is reduced.

In additional embodiments, the invention relates to a method for treating a neurodegenerative disease, comprising: a) providing; i) a patient, wherein said patient has a neurodegenerative disease characterized by abnormally low dopamine levels; ii) a composition consisting essentially of a methyl-substituted tetrahydropyridine conjugated to a selenium antioxidant; b) administering said composition to said patient under conditions such that said dopamine levels are increased.

In some embodiments, the invention relates to a method of managing, preventing or treating neurodegenerative diseases comprising: providing i) a subject with symptoms of a neurodegenerative disease and a therapeutic composition comprising an antioxidant disclosed herein and ii) administering said composition to said subject. In further embodiments, said subject is a human. In further embodiments, said neurodegenerative disease is selected from the group consisting of cancer, Parkinson's, Alzheimer's, and multiple sclerosis. In further embodiments, said compounds are administered in combination with one or more current therapeutics such as L-DOPA, selegiline, desmethylselegiline, tocopherol, riluzole, interferon, cyclosporine, azathioprine, methotrexate, haloperidol, risperidone, olanzapine, and quetiapine or combinations thereof.

In some embodiments, the invention relates to compositions comprising compounds disclosed herein and derivatives thereof. In some embodiments, the invention relates to a composition comprising a conjugate comprising an antioxidant moiety and a targeting moiety. In additional embodiments, the invention relates to compounds disclosed herein neuroprotective of hydrogen peroxide and superoxide.

In some embodiments, the invention relates to a conjugate with peroxidase and/or superoxide dismutase activity comprising an antioxidant moiety and a pyridinium cationic moiety. In additional embodiments, the invention relates to a conjugate with peroxidase and/or superoxide dismutase activity comprising an antioxidant moiety and a hydropyridine moiety wherein mixing said conjugate with type B monoamine oxidase (MAO-B) provides a conjugate comprising antioxidant moiety and a pyridinium cationic moiety. In further embodiments, said antioxidant moiety is selected from a molecular arrangement comprising selenium atom and nitroxide moiety. In further embodiments, said antioxidant moiety is selected from a Mn-Salen moiety, a tetraalkylheterocyclic nitroxide moiety, a heterocyclic selenium moiety, and a heterocyclic selenium nitroxide moiety. In further embodiments, said hydropyridine moiety is the molecular arrangement of the following formula:

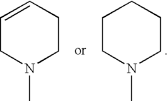

In further embodiments, said Mn-salen moiety is the molecular arrangement of the following formula:

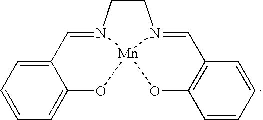

In further embodiments, said hydropyridine moiety and said Mn-salen moiety are connected by a linking group providing a conjugate comprising the following formula:

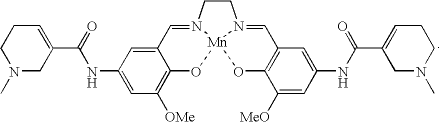

In further embodiments, said tetraalkylheterocyclic nitroxide moiety is the molecular arrangement of the following formula:

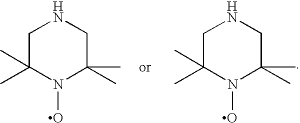

In further embodiments, said hydropyridine moiety and said tetraalkylheterocyclic nitroxide moiety are connected by a molecular arrangement providing a conjugate comprising the following formula:

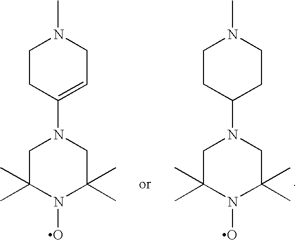

In further embodiments, said heterocyclic selenium moiety is the molecular arrangement of the following formula:

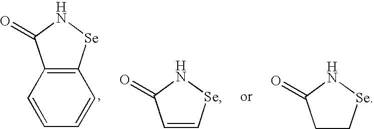

In further embodiments, said hydropyridine moiety and said heterocyclic selenium moiety are connected by a molecular arrangement providing a conjugate comprising the following formula:

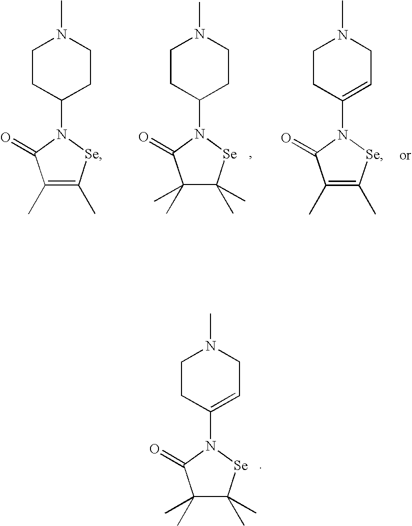

In further embodiments, said heterocyclic selenium nitroxide moiety is the molecular arrangement of the following formula selected from the group consisting of:

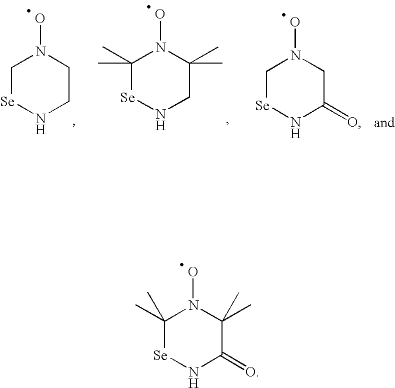

In further embodiments, said hydropyridine moiety and said heterocyclic selenium nitroxide moiety are connected by a molecular arrangement providing a conjugate comprising the following formula selected from the group consisting of

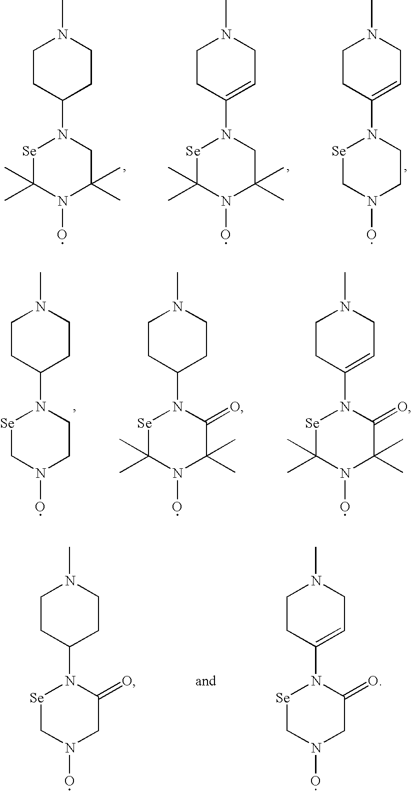

In some embodiments, the invention relates to a compound having the following formula: 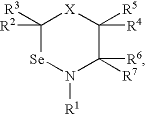
or pharmaceutically acceptable salts thereof wherein, R¹ is hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 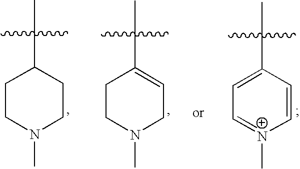
R² and R³ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 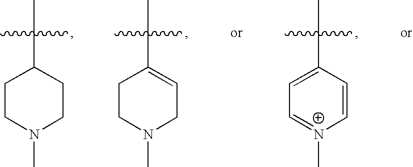
R² and R³ are ═O, ═S, ═NH, ═N—R⁸, ═N—OR⁸, ═N—SR⁸, ═N—NR⁸R⁹ or two hydrogens each independently bonded to the carbon by a single bond; R⁴ and R⁵ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 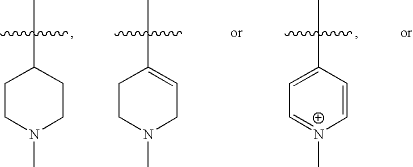
R⁴ and R⁵ are ═O, ═S, ═NH, ═N—R⁸, ═N—OR⁸, ═N—SR⁸, ═N—NR⁸R⁹ or two hydrogens each independently bonded to the carbon by a single bond; R⁶ and R⁷ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 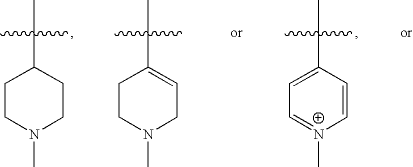
R⁶ and R⁷ are ═O, ═S, ═NH, ═N—R⁸, ═N—OR⁸, ═N—SR⁸, ═N—NR⁸R⁹ or two hydrogens each independently bonded to the carbon by a single bond; or R⁵ and R⁶ are absent and the carbons to which R⁵ and R⁶ are attached form a double bond and R⁴ and R⁷ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 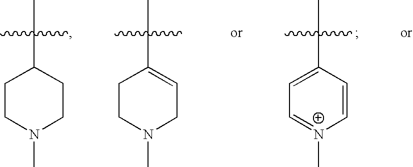
R⁶ is absent and the carbon to which R⁶ is attached and the nitrogen attached to the carbon to which R⁶ is attached forms a double bond and R⁷ is hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 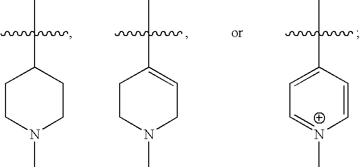
R⁸ and R⁹ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl; and X is °N—O, N—OH, or N═O.

In additional embodiments, the invention relates to a compound having the following formula: 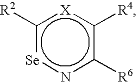
or pharmaceutically acceptable salt thereof wherein, R², R⁴, and R⁶ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 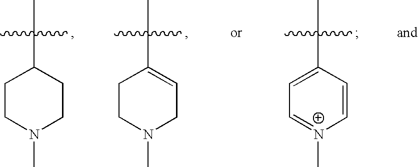
X is N—O°, or N—OH.

In some embodiments, the invention relates to a compound having the following formula: 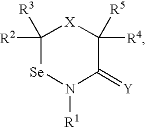
or pharmaceutically acceptable salts thereof wherein, Y is ═O, ═S, ═NH, ═N—R⁶, N—OR⁶, ═N—SR⁶, ═N—NR⁸R⁸ or two hydrogens each independently bonded to the carbon by a single bond; R¹, R², R³, R⁴, R⁵, R⁶, and R⁷ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 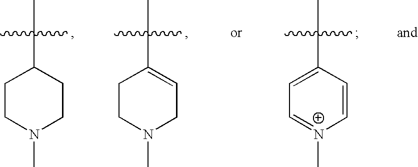
X is N—O°, N—OH, or N═O.

With regard to certain embodiments, a chemical structure may be drawn with two lines between a first atom and substituent and meaning that there are two bonds, i.e., designate a double between the first atom and a defined substituent or it may designate two single bonds between the first atom and two defined substituent atoms.

In additional embodiments, the invention relates to a compound having the following formula: 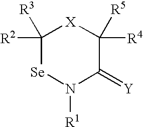
or pharmaceutically acceptable salts thereof wherein, R¹ is 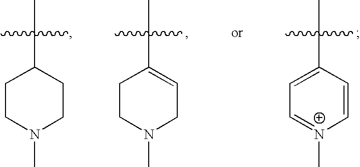
Y is ═O, ═S, ═NH, ═N—R⁶, ═N—OR⁶, ═N—SR⁶, ═N—NR⁶R⁷ or two hydrogens each independently bonded to the carbon by a single bond; R², R³, R⁴, R⁵ are the same or different and, at each occurrence, independently hydrogen and alkyl; and X is N—O°, N—OH, or N═O.

With regard to certain embodiments, a chemical structure may be drawn with two lines between a first atom and substituent and meaning that there are two bonds, i.e., designate a double between the first atom and a defined substituent or it may designate two single bonds between the first atom and two defined substituent atoms.

In some embodiments, the invention relates to a compound having the following formula: 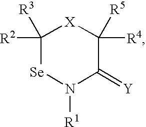
or pharmaceutically acceptable salts thereof wherein Y is ═O, ═S, ═NH, ═N—R⁶, N—OR⁶, ═N—SR⁶, ═N—NR⁶R⁷ or two hydrogens each independently bonded to the carbon by a single bond; R¹ is the group 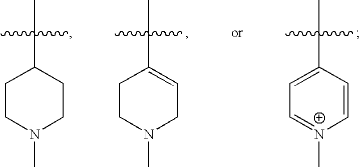
R², R³, R⁴, R⁵, R⁶, and R⁷ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl; and X is N—O°, N—OH, or N═O.

In additional embodiments, the invention relates to a compound having the following formula: 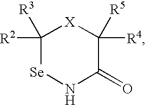
or salts thereof wherein, R², R³, R⁴, and R⁵ are the same or different and, at each occurrence, independently hydrogen and alkyl; and X is N—O°, N—OH, or N═O.

In some embodiments, the invention relates to a compound having the following formula: 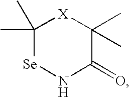
or salts thereof wherein, X is N—O°, N—OH, or N═O.

In some embodiments, the invention relates to a compound having the following formula: 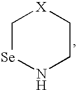
or salts thereof wherein, X is N—O°, N—OH, or N═O.

In some embodiments, the invention relates to a composition comprising a compound selected from the group consisting of 1,4-diaza-6-oxo-2-selenacyclohexan-4-oxyls, N²-(selenylmethyl)-N²-hydroxyglycinamide, N²-(bromomethyl)-N²-hydroxyglycinamide, N²-[(2,4-dinitrophenoxy)methyl]-N²-hydroxyglycinamide, [[(2,4-dinitrophenoxy)methyl] (hydroxy)amino]acetonitrile, 1-bromo-N-[(2,4-dinitrophenoxy)methyl]-N-hydroxy methanamine, and 1-bromo-N-(bromomethyl)-N-hydroxymethanamine

In some embodiments, the invention relates to a method of making 1-bromo-N-(bromomethyl)-N-hydroxymethanamine comprising providing hydroxylamine and dibromomethane and mixing hydroxylamine and dibromomethane under conditions such that 1-bromo-N-(bromomethyl)-N-hydroxymethanamine is formed.

In some embodiments, the invention relates to a method of making 1-bromo-N-[(2,4-dinitrophenoxy)methyl]-N-hydroxymethanamine comprising providing 1-bromo-N-(bromomethyl)-N-hydroxymethanamine and 2,4-dinitrophenol and mixing hydroxymethanamine and 2,4-dinitrophenol under conditions such that 1-bromo-N-[(2,4-dinitrophenoxy)methyl]-N-hydroxymethanamine is formed.

In some embodiments, the invention relates to a method of making [[(2,4-dinitrophenoxy)methyl](hydroxy)amino]acetonitrile comprising providing 1-bromo-N-[(2,4-dinitrophenoxy)methyl]-N-hydroxymethanamine and cyanide ion and mixing 1-bromo-N-[(2,4-dinitrophenoxy)methyl]-N-hydroxymethanamine and cyanide ion under conditions such that [[(2,4-dinitrophenoxy)methyl](hydroxy)amino]acetonitrile is formed.

In some embodiments, the invention relates to a method of making N²-[(2,4-dinitrophenoxy)methyl]-N²-hydroxyglycinamide comprising providing [[(2,4-dinitrophenoxy)methyl](hydroxy)amino]acetonitrile and hydroxide ion and mixing [[(2,4-dinitrophenoxy)methyl](hydroxy)amino]acetonitrile and hydroxide ion under conditions such that N²-[(2,4-dinitrophenoxy)methyl]-N²-hydroxyglycinamide is formed.

In some embodiments, the invention relates to a method of making N²-(bromomethyl)-N²-hydroxyglycinamide comprising providing N²-[(2,4-dinitrophenoxy)methyl]-N²-hydroxyglycinamide and bromide ion and mixing N²-[(2,4-dinitrophenoxy)methyl]-N²-hydroxyglycinamide and bromide ion under conditions such that N²-(bromomethyl)-N²-hydroxyglycinamide is formed.

In some embodiments, the invention relates to a method of making N²-(selenylmethyl)-N²-hydroxyglycinamide comprising providing N²-(bromomethyl)-N²-hydroxyglycinamide and sodium selenide and mixing N²-(bromomethyl)-N²-hydroxyglycinamide and sodium selenide under conditions such that N²-(selenylmethyl)-N²-hydroxyglycinamide is formed.

In some embodiments, the invention relates to a method of making 1,4-diaza-6-oxo-2-selenacyclohexan-4-oxyl comprising providing N²-(selenylmethyl)-N²-hydroxyglycinamide and hydrogen peroxide under conditions such that 1,4-diaza-6-oxo-2-selenacyclohexan-4-oxyl is formed.

In some embodiments, the invention relates to a composition comprising a compound selected from the group consisting 1,4-diaza-2-selenacyclohexan-4-oxyl, N-(bromomethyl)-N′-(1,3-dimethylbutylidene)-N-hydroxyethane-1,2-diamine, [(2-{[1,3-dimethylbutylidene]amino}ethyl)(hydroxy)amino]methaneselenol, and N-(1,3-dimethylbutylidene)-N′-hydroxyethane-1,2-diamine.

In some embodiments, the invention relates to a method of making 1,4-diaza-2-selenacyclohexan-4-oxyl comprising providing [(2-{[1,3-dimethylbutylidene]amino}ethyl)(hydroxy)amino]methaneselenol and water under conditions such that 1,4-diaza-2-selenacyclohexan-4-oxyl is formed.

In some embodiments, the invention relates to a method of making [(2-{[1,3-dimethylbutylidene]amino}ethyl)(hydroxy)amino]methaneselenol comprising providing N-(bromomethyl)-N′-(1,3-dimethylbutylidene)-N-hydroxyethane-1,2-diamine and Selenium under conditions such that [(2-{[1,3-dimethylbutylidene]amino}ethyl)(hydroxy)amino]methaneselenol or salt thereof is formed.

In some embodiments, the invention relates to a method of making N-(bromomethyl)-N′-(1,3-dimethylbutylidene)-N-hydroxyethane-1,2-diamine comprising providing dibromomethane and N-(1,3-dimethylbutylidene)-N′-hydroxyethane-1,2-diamine and mixing N-(1,3-dimethylbutylidene)-N′-hydroxyethane-1,2-diamine and dibromomethane under conditions such that N-(bromomethyl)-N′-(1,3-dimethylbutylidene)-N-hydroxyethane-1,2-diamine is formed.

In some embodiments, the invention relates to a method of making N-(1,3-dimethylbutylidene)-N′-hydroxyethane-1,2-diamine comprising providing hydroxyamine and 2-bromo-N-(1,3-dimethylbutylidene)ethanamine and mixing 2-bromo-N-(1,3-dimethylbutylidene)ethanamine and hydroxyamine under conditions such that N-(1,3-dimethylbutylidene)-N′-hydroxyethane-1,2-diamine is formed.

In some embodiments, the invention relates to a compound having the following formula: 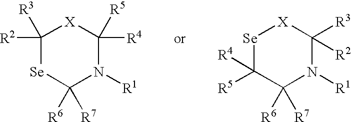
or pharmaceutically acceptable salts thereof wherein, R¹ is hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 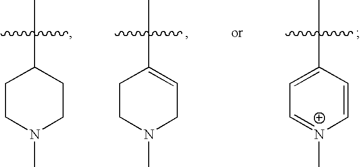
R² and R³ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 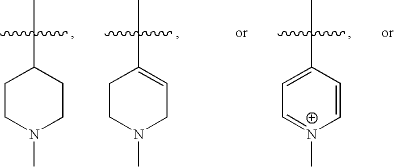
R² and R³ are ═O, ═S, ═Se, ═NH, ═N—R⁸, ═N—OR⁸, ═N—SR⁸, ═N—SeR⁸, or ═N—NR⁸R⁹; R⁴ and R⁵ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 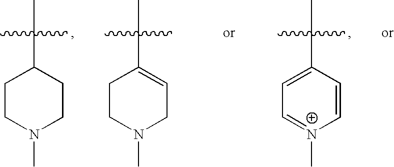
R⁴ and R⁵ are ═O, ═S, ═Se, ═NH, ═N—R⁷, ═N—OR⁸, ═N—SR⁸, ═N—SeR⁸, or ═N—NR⁹R⁸; R⁶ and R⁷ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 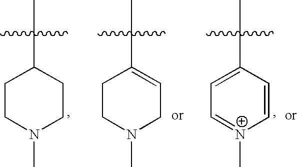
R⁶ and R⁷ are ═O, ═S, ═Se, ═NH, ═N—R⁸, ═N—OR⁸, ═N—SR⁸, ═N—SeR⁸, or ═N—NR⁸R⁹; or R⁵ and R⁶ are absent and the carbons to which R⁵ and R⁶ are attached form a double bond and R⁴ and R⁷ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 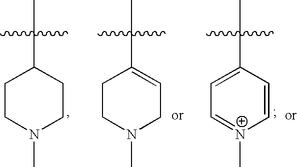
R⁶ is absent and the carbon to which R⁶ is attached and the nitrogen attached to the carbon to which R⁶ is attached forms a double bond and R⁷ is hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 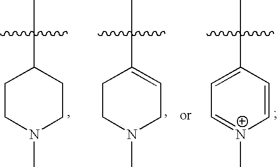
R⁸ and R⁹ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl; and X is N—O°, N—OH, or N═O.

In additional embodiments, the invention relates to a compound having the following formula: 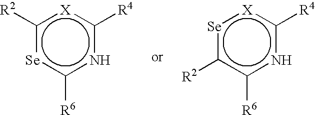
or pharmaceutically acceptable salt thereof wherein, R², R⁴, and R⁶ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 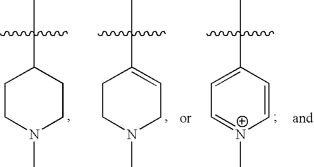
; X is N—O° or N—OH.

In some embodiments, the invention relates to a compound having the following formula: 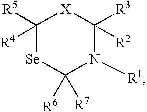
or salts thereof wherein, R¹ is hydrogen, 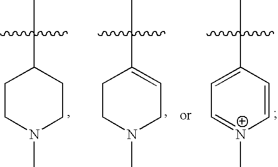
R², R³, R⁴, R⁵, R⁶, and R⁷ are the same or different and, at each occurrence, independently hydrogen or alkyl; and X is N—O°, N—OH, or N═O.

In one embodiment, the invention relates to a method providing a compound comprising a nitroxide and a hydropyridine moiety and using said compound as a free radical magnetic resonance imaging (MRI) agent.

In some embodiments, the invention relates to a method of detecting free radical nitroxide in mammalian brain tissue comprising a) providing i) a subject comprising mammalian brain tissue, ii) a compound comprising a free radical nitroxide and substituted tetra-hydro, methyl-pyridine moiety, and iii) a magnetic resonance spectrometer configured to detect electron spin resonance (ESR) of said free radical nitroxide moiety or the localized changes in the spin properties of nearby nuclear spins by said radical; b) administering said compound to said subject; c) detecting said electron spin resonance with said magnetic resonance spectrometer in said brain tissue.

In some embodiments, the invention relates to a method comprising using a compound comprising a nitroxide and hydropyridine moiety as a free radical MRI agent. In additional embodiments, the invention relates to a method of detecting free radical nitroxide in mammalian brain tissue comprising: a) providing; i) a subject comprising mammalian brain tissue, ii) a compound comprising a free radical nitroxide and hydropyridine moiety, and iii) a magnetic resonance spectrometer configured to detect electron spin resonance of said free radical nitroxide moiety; b) administering said compound to said subject; c) detecting said electron spin resonance with said magnetic resonance spectrometer in said brain tissue. In further embodiments, said brain tissue comprises a dopaminergic neuron. In further embodiments, said compound further comprises a selenium atom. In further embodiments, said compound comprising a nitroxide and hydropyridine moiety is selected from the group consisting of: 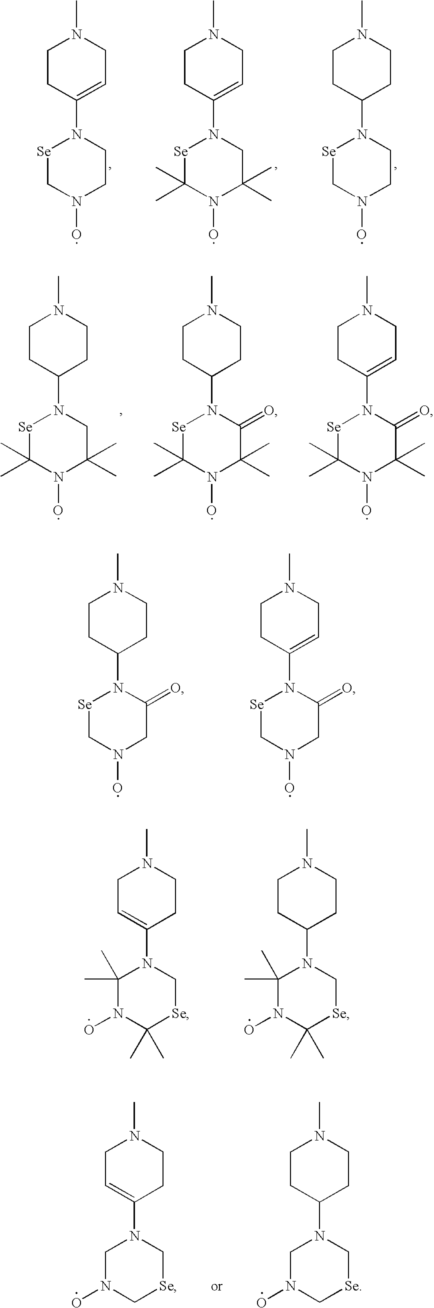
In further embodiments, said compound comprising a nitroxide and hydropyridine moiety is selected from the group consisting of:

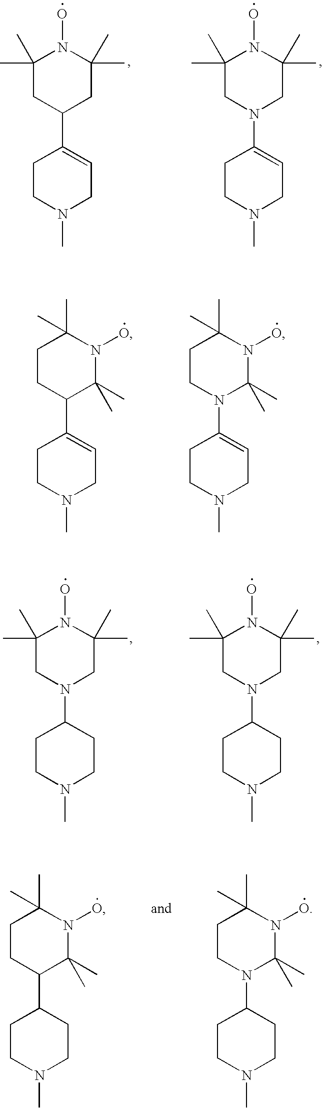

In certain embodiments the invention relates to a method of imaging mammalian brain tissue comprising a) providing i) a subject comprising mammalian brain tissue, ii) a composition comprising an compound comprising selenium atom and a tetrahydropyridine moeity, and iii) a magnetic resonance spectrometer configured to detect electron spin resonance of selenium isotope 77, b) administering said compound to said subject; c) detecting said electron spin resonance with said magnetic resonance spectrometer in said brain tissue. In further embodiments, said composition comprising said selenium atom is enriched in selenium isotope 77. In further embodiments, said compound has the following formula: 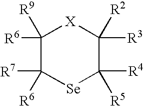
or salts thereof wherein, R², R³, R⁴, R⁵, R⁶, R⁷R⁸, and R⁹ are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl; X is N—R¹; and R¹ is 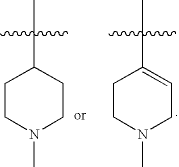
In further embodiments, said compound further comprises a free radical nitroxide group.

In further embodiments, the invention relates to compounds and methods of making compounds disclosed herein.

In some embodiments, the invention relates to a compound having the following formula:

In some embodiments, the invention relates to a compound having the following formula: 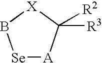
or salts thereof wherein, A is —CR⁴R⁵—, -Z-CR⁶R⁷—, —CR⁴R⁵—CR⁶R⁷—, or -Z-CR⁴R⁵—CR⁶R⁷—; B is a single bond, —CR⁴R⁵—, or —CR⁶R⁷—CR⁸R⁹—; R², R³, R⁴, R⁵, R⁶, R⁷ R⁸, and R⁹ are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl; X and Z are the same or different and, at each occurrence, independently N—R¹, N—O°, N—OH, or N═O; and R¹ is hydrogen,

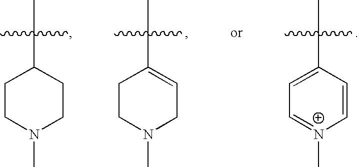

In some embodiments, the invention relates to a compound having the following formula: 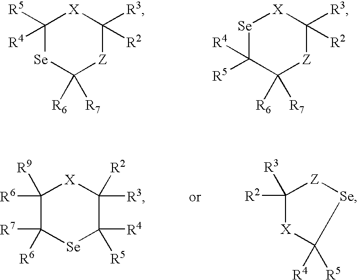
or salts thereof wherein, R², R³, R⁴, R⁵, R⁶, R⁷ R⁸, and R⁹ are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl; X and Z are the same or different and, at each occurrence, independently N—R¹, N—O°, N—OH, or N═O; and R¹ is hydrogen,

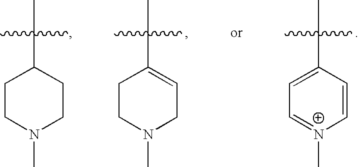

In some embodiments, the invention relates to a compound of the formula: 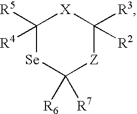
or salts thereof wherein, R², R³, R⁴, R⁵, R⁶, and R⁷ are the same or different and, at each occurrence, independently hydrogen, alkyl, or substituted alkyl; X and Z are the same or different and, at each occurrence, independently N—R¹, N—O°, N—OH, or N═O; and R¹ is hydrogen,

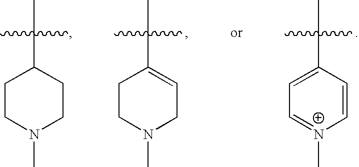

In some embodiments, the invention relates to a compound of the formula: 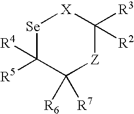
or salts thereof wherein, R², R³, R¹, R⁴, R⁵, R⁶ are the same or different and, at each occurrence, independently hydrogen, alkyl, substituted alkyl; X and Z are the same or different and, at each occurrence, independently N—R¹, N—O°, N—OH, or N═O; and R¹ is hydrogen,

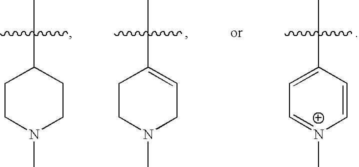

In some embodiments, the invention relates to a compound of the formula: 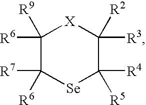
or salts thereof wherein, R², R³, R⁴, R⁵, R⁶, R⁷ R⁸, and R⁹ are the same or different and, at each occurrence, independently hydrogen, alkyl, substituted alkyl; X is N—R¹, N—O°, N—OH, or N═O; and R¹ is hydrogen,

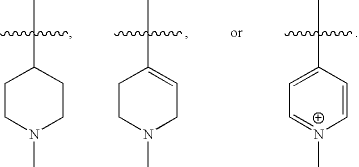

In some embodiments, the invention relates to a compound of the formula: 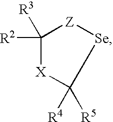
or salts thereof wherein, R², R³, R⁴, R⁵ are the same or different and, at each occurrence, independently hydrogen, alkyl, substituted alkyl; X and Z are the same or different and, at each occurrence, independently N—R¹, N—O°, N—OH, or N═O; and R¹ is hydrogen,

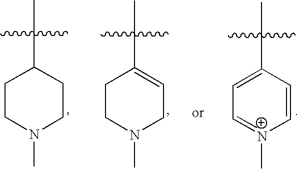

In some embodiments, the invention relates to a compound of the formula: 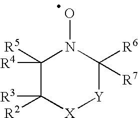
and salts thereof wherein, X and Y are the same or different and, at each occurrence, independently —CR⁸R⁹—, CH—R¹, or N—R¹, provided that at least one of the group X and Y is N—R¹; R¹, R², R³, R⁴, R⁵, R⁶, R⁷ R⁸, and R⁹ are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl; and R¹ is hydrogen,

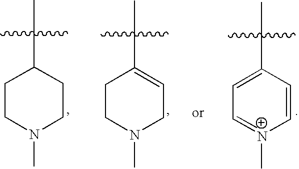

In further embodiments, X is CH—R¹; Y is —CR⁸R⁹—, and R², R³, R⁴, R⁵, R⁶, R⁷ R⁸, and R⁹ are the same or different and, at each occurrence, independently hydrogen, alkyl, or substituted alkyl.

In some embodiments, the invention relates to a method of detecting free radical nitroxide in mammalian brain tissue comprising a) providing i) a subject comprising mammalian brain tissue, ii) a compound comprising a free radical nitroxide and tetrahydropyridine moiety, and iii) a magnetic resonance spectrometer configured to detect electron spin resonance of said free radical nitroxide moiety; b) administering said compound to said subject; c) detecting said electron spin resonance with said magnetic resonance spectrometer in said brain tissue. In further embodiments, said tetrahydropyridine moiety is an N-methyl substituted tetrahydropyridine. In further embodiments, said detection occurs by measuring nuclear spins and correlating those to the presence of free radical nitroxide in said tissue. In further embodiments, said nuclear spins are from water molecules. In further embodiments, said brain tissue comprises a dopaminergic neuron. In further embodiments, said compound has the following formula: 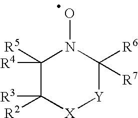
and salts thereof wherein, X and Y are the same or different and, at each occurrence, independently —CR⁸R⁹—, CH—R¹, or N—R¹, provided that at least one of the group X and Y is N—R¹; R², R³, R⁴, R⁵, R⁶, R⁷ R⁸, and R⁹ are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl; and R¹ is hydrogen, 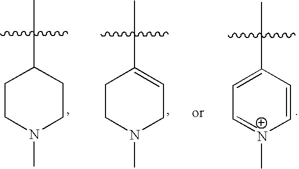
In further embodiments, said compound further comprises a selenium atom. In further embodiments, said compound has the following formula: 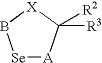
or salts thereof wherein, A is —CR⁴R⁵—, -Z-CR⁶R⁷—, —CR⁴R⁵—CR⁶R⁷—, or -Z-CR⁴R⁵—CR⁶R⁷—; B is a single bond, —CR⁴R⁵—, or —CR⁶R⁷—CR⁸R⁹—; R², R³, R⁴, R⁵, R⁶, R⁷ R⁸, and R⁹ are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl; X and Z are the same or different and, at each occurrence, independently N—R¹, N—O°, N—OH, or N═O, provided that at least one of the group X and Z is N—R¹ and at least one or the group X and Z is N—O°; and R¹ is hydrogen,

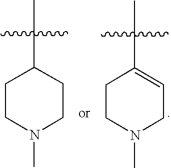

In other embodiments, the invention relates to a method of imaging mammalian brain tissue comprising a) providing i) a subject comprising mammalian brain tissue, ii) a composition comprising an conjugate comprising an atomic isotope with nuclear spin and a hydropyridine moiety, and iii) a magnetic resonance spectrometer configured to detect nuclear spin resonance of said atomic isotope; b) administering said compound to said subject; c) detecting said nuclear spin resonance with said magnetic resonance spectrometer in said brain tissue; and d) imaging said brain tissue. In further embodiments, said composition is enriched in said atomic isotope, i.e., in a concentration greater than natural abundance.

In other embodiments, the invention relates to a method of imaging mammalian brain tissue comprising a) providing i) a subject comprising mammalian brain tissue, ii) a composition comprising an compound comprising selenium atom and a tetrahydropyridine moiety, and iii) a magnetic resonance spectrometer configured to detect nuclear spin resonance of selenium isotope 77; b) administering said compound to said subject; c) detecting said nuclear spin resonance with said magnetic resonance spectrometer in said brain tissue; and d) imaging said brain tissue. In further embodiments, said tetrahydropyridine is an N-methyl substituted tetrahydropyridine. In further embodiments, said detecting is by measuring nuclear spin resonance. In further embodiments said nuclear spin is selenium isotope 77. In further embodiments, said composition comprising said selenium atom is enriched in selenium isotope 77. In further embodiments, said compound has the following formula: 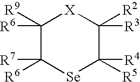
or salts thereof wherein, R², R³, R⁴, R⁵, R⁶, R⁷ R⁸, and R⁹ are the same or different and, at each occurrence, independently hydrogen, alkyl, substituted alkyl; X is N—R¹; and R¹ is 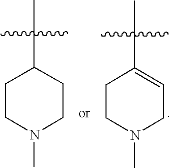
In further embodiments, said compound further comprises a free radical nitroxide group. In further embodiments, said compound has the following formula: 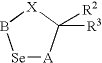
or salts thereof wherein, A is —CR⁴R⁵—, -Z-CR⁶R⁷—, —CR⁴R⁵—CR⁶R⁷—, or -Z-CR⁴R⁵—CR⁶R⁷—; B is a single bond, —CR⁴R⁵—, or —CR⁶R⁷—CR⁸R⁹—; R², R³, R⁴, R⁵, R⁶, R⁷ R⁸, and R⁹ are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl; X and Z are the same or different and, at each occurrence, independently N—R¹, N—O°, N—OH, or N═O, provided that at least one of the group X and Z is N—R¹ and at least one or the group X and Z is N—O°; and R¹ is hydrogen,

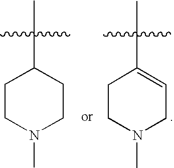

In further embodiments, the invention relates to a method of making a compound comprising: providing: a compound having the following structure: 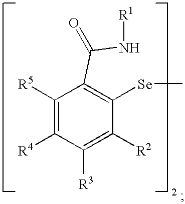
and a reducing agent and mixing said compound and reducing agent under conditions such that a compound having the following formula is formed: 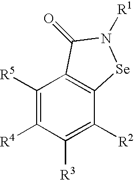
and salts thereof wherein R¹ is alkyl, substituted alkyl, aryl, substituted aryl, arylalkyl, substituted arylalkyl, heterocycle, substituted heterocycle, heterocyclealkyl, or substituted heterocyclealkyl; R², R³, R⁴, R⁵, are the same or different and, at each occurrence, independently hydrogen, halogen, alkyl, substituted alkyl, aryl, substituted aryl, arylalkyl, substituted arylalkyl, heterocycle, substituted heterocycle, heterocyclealkyl, substituted heterocyclealkyl or other substituent. In further embodiments, said reducing agent is dithiothreitol

In some embodiments, the invention relates to a method of making a compound comprising: providing: a compound having the following structure: 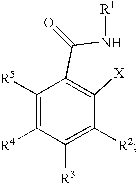
and salts thereof wherein X is Br or I; MSeH wherein M is a metal; and mixing said compound and MSeH under conditions such that a compound having the following formula is formed: 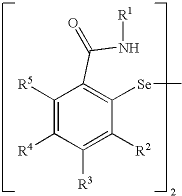
and salts thereof wherein R¹ is alkyl, substituted alkyl, aryl, substituted aryl, arylalkyl, substituted arylalkyl, heterocycle, substituted heterocycle, heterocyclealkyl, or substituted heterocyclealkyl; R², R³, R⁴, R⁵, are the same or different and, at each occurrence, independently hydrogen, halogen, alkyl, substituted alkyl, aryl, substituted aryl, arylalkyl, substituted arylalkyl, heterocycle, substituted heterocycle, heterocyclealkyl, substituted heterocyclealkyl or other substituent. In further embodiments, M is Na.

In some embodiments, the invention relates to a compound having the following formula: 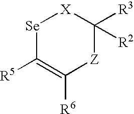
or salts thereof wherein, R², R³, are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl; R⁵ and R⁶ and atoms to which they are attached form a five, six or seven atom aromatic or nonaromatic ring; X and Z are the same or different and, at each occurrence, independently N—R¹, N—O°, N—OH, or N═O; and R¹ is hydrogen

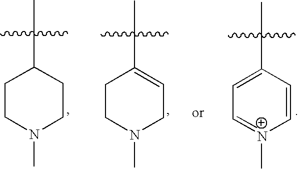

In some embodiments, the invention relates to a compound having the following formula: 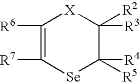
or salts thereof wherein, R², R³, R⁴, and R⁵ are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl; R⁶ and R⁷ and atoms to which they are attached form a five, six or seven atom aromatic or nonaromatic ring; X and Z are the same or different and, at each occurrence, independently N—R¹, N—O°, N—OH, or N═O; and R¹ is hydrogen

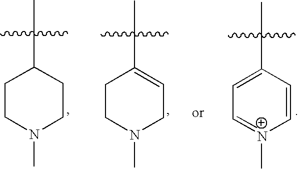

In some embodiments, the invention relates to a compound having the following formula: 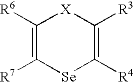
R³ and R⁴ and atoms to which they are attached form a five, six or seven atom aromatic or nonaromatic ring; R⁶ and R⁷ and atoms to which they are attached form a five, six or seven atom aromatic or nonaromatic ring; X is N—R¹, N—O°, N—OH, or N═O; and R¹ is hydrogen

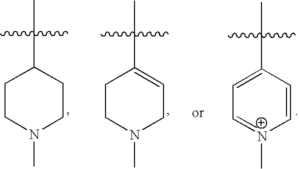

In some embodiments, the invention relates to a compound having the following formula: 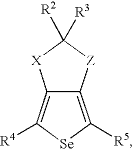
or salts thereof wherein, R², R³, R⁴, and R⁵ are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl; X and Z are the same or different and, at each occurrence, independently hydrogen, N—R¹, N—O°, N—OH, or N═O; and R¹ is hydrogen,

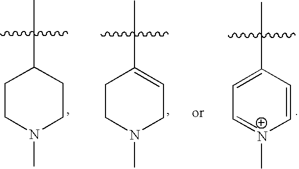

In some embodiments, the invention relates to a composition comprising compounds disclosed herein with a selenium 77 isotope content that is greater than found in natural elemental compositions of selenium metal, i.e., greater than natural abundance, and there use in MRI imaging.

In some embodiments the invention relates to the use by administration of pharmaceutical composition comprising compounds disclosed herein for treating, preventing, and/or managing a subject diagnosed with or at risk for acute as well as chronic neurological disorders, including head injury, stroke (both ischemic and hemorrhagic), and spinal cord trauma. In further embodiments, said pharmaceutical formulations are preferably parenteral drug formulations for acute injury and oral formulations for chronic indications.

In some embodiments the invention relates to the use by administration of pharmaceutical composition comprising compounds disclosed herein for treating, preventing, and/or managing a subject diagnosed with or at risk for chronic and acute inflammatory conditions.

In some embodiments, the invention relates to compounds having the following formulas: 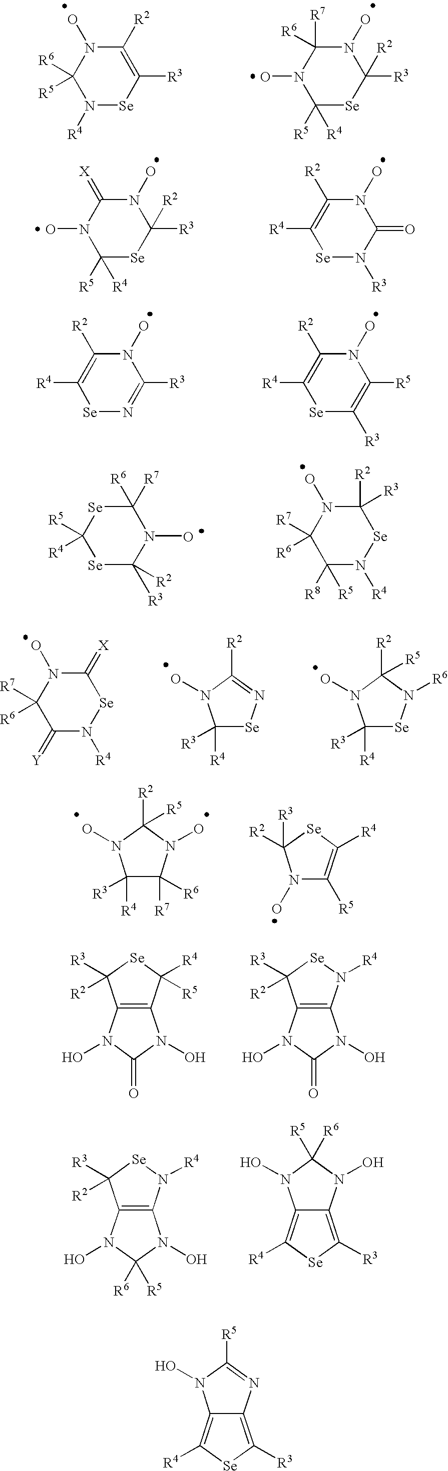
wherein R², R³, R⁴, R⁵, R⁶, and R⁷ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, or the groups 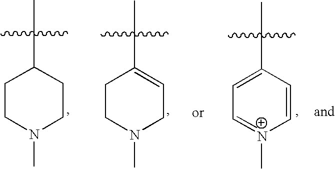
X and Y are the same or different and, at each occurrence, independently ═O, ═S, ═NH, ═N—R⁶, ═N—OR⁶, ═N—S⁶, ═N—NR⁶R⁷.

In some embodiments, the invention relates to a compound having the following formula: 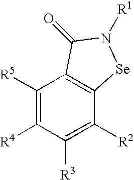
and salts thereof wherein R², R³, R⁴, and R⁵ are the same or different and, at each occurrence, independently hydrogen, alkyl, substituted alkyl, aryl, substituted aryl, arylalkyl, substituted arylalkyl, heterocycle, substituted heterocycle, heterocyclealkyl, substituted heterocyclealkyl, R¹ is selected from the groups

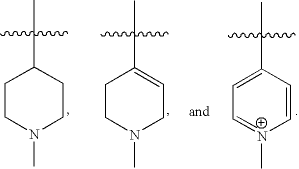

In some embodiments, the invention relates to treating, preventing, or managing a disease and/or conduction preferably related to a neurological dysfunction by administering a pharmaceutical composition with a compound disclosed herein to a patient with, at risk for, or diagnosed with said condition or disease. In further embodiments, said conditions include Absence of the Septum Pellucidum, Acid Lipase Disease, Acquired Epileptiform Aphasia, Acute Disseminated Encephalomyelitis, ADHD, Adie's Pupil, Adie's Syndrome, Adrenoleukodystrophy, Agenesis of the Corpus Callosum, Agnosia, Aicardi Syndrome, AIDS-Neurological Complications, Alexander Disease, Alpers' Disease, Alternating Hemiplegia, Alzheimer's Disease, Amyotrophic Lateral Sclerosis, Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar/Spinocerebellar Degeneration, Attention Deficit-Hyperactivity Disorder, Autism, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysm, Cerebral Arteriosclerosis, Cerebral Atrophy, Cerebral Beriberi, Cerebral Gigantism, Cerebral Hypoxia, Cerebral Palsy, Cerebro-Oculo-Facio-Skeletal Syndrome, Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, COFS, Colpocephaly, Coma and Persistent Vegetative State, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Deep Brain Stimulation for Parkinson's Disease, Dejerine-Klumpke Palsy, Dementia, Dementia-Multi-Infarct, Dementia-Semantic, Dementia-Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalotrigeminal Angiomatosis, Epilepsy, Erb-Duchenne and Dejerine-Klumpke Palsies, Erb's Palsy, Essential Tremor, Extrapontine Myelinolysis, Fabry's Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fisher Syndrome, Floppy Infant Syndrome, Friedreich's Ataxia, Frontotemporal Dementia, Gangliosidoses, Gaucher's Disease, Gerstmann's Syndrome, Gerstmann-Straussler-Scheinker Disease, Giant Cell Arteritis, Giant Cell Inclusion Disease, Globoid Cell Leukodystrophy, Glossopharyngeal Neuralgia, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus-Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathy, Iniencephaly, Intestinal Lipodystrophy, Intracranial Cysts, Intracranial Hypertension, Isaac's Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Klüver-Bucy Syndrome, Korsakoffs Amnesic Syndrome, Krabbe Disease, Kugelberg-Welander Disease, Kuru, Lambert-Eaton Myasthenic Syndrome, Landau-Kleffer Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lissencephaly, Locked-In Syndrome, Lou Gehrig's Disease, Lupus—Neurological Sequelae, Lyme Disease—Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini-Strokes, Mitochondrial Myopathies, Mobius Syndrome, Monomelic Amyotrophy, Motor Neuron Diseases, Moyamoya Disease, Mucolipidoses, Mucopolysaccharidoses, Multifocal Motor Neuropathy, Multi-Infarct Dementia, Multiple Sclerosis, Multiple System Atrophy, Multiple System Atrophy with Orthostatic Hypotension, Muscular Dystrophy, Myasthenia-Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications Of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy-Hereditary, Neurosarcoidosis, Neurotoxicity, Nevus Cavernosus, Niemann-Pick Disease, Normal Pressure Hydrocephalus, Occipital Neuralgia, Occult Spinal Dysraphism Sequence, Ohtahara Syndrome, Olivopontocerebellar Atrophy, Opsoclonus Myoclonus, Orthostatic Hypotension, O'Sullivan-McLeod Syndrome, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Postherpetic Neuralgia, Postinfectious Encephalomyelitis, Post-Polio Syndrome, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudotumor Cerebri, Ramsay Hunt Syndrome I, Ramsay Hunt Syndrome II, Rasmussen's Encephalitis, Reflex Sympathetic Dystrophy Syndrome, Refsum Disease, Refsum Disease-Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, SUNCT Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Tuberous Sclerosis, Vascular Erectile Tumor, Vasculitis including Temporal Arteritis, Von Economo's Disease, Von Hippel-Lindau Disease (VHL), Von Recklinghausen's Disease, Wallenberg's Syndrome, Werdnig-Hoffman Disease, Wemicke-Korsakoff Syndrome, West Syndrome, Whiplash, Whipple's Disease, Williams Syndrome, Wilson's Disease, Wolman's Disease, X-Linked Spinal and Bulbar Muscular Atrophy, or Zellweger Syndrome.

In some embodiments, the invention relates to treating, preventing, or managing a oxidative injury by administering a pharmaceutical composition with a compound disclosed herein to a patient with, at risk for, or diagnosed with said injury. Oxidative injury occurs in a variety of organs, for example, but not limited to, ischemic myocardium from injury during bypass, and the brain from hemorrhagic stroke.

### BRIEF DESCRIPTION OF THE FIGURES

FIG. 1 illustrates the interaction of reactive oxygen species and reactive nitrogen species with physiological components.

FIG. 2 illustrates the reversible oxidation and reduction of EUK-134.

FIG. 3 illustrates the reversible oxidation and reduction of TEMPO.

FIG. 4 illustrates the reversible oxidation and reduction of Ebselen.

FIG. 5 illustrates the reversible oxidation and reduction of one embodiment of a heterocyclic selenium nitroxide.

FIG. 6 illustrates the migration and physiological processing of MPTP to the active metabolite MMP, and transport into a dopaminergic neuron.

FIG. 7 illustrates several embodiments of the current invention including potential physiological processing of them by MOA-B into active metabolites.

FIG. 8 illustrates the reversible oxidation and reduction of one embodiment of a heterocyclic selenium nitroxide containing a pyridinium cationic moiety.

FIG. 9 shows a synthetic scheme for making embodiments of the current invention.

FIG. 10A shows a synthetic scheme for making embodiments of the current invention.

FIG. 10B shows a synthetic scheme for making embodiments of the current invention.

FIG. 11 shows a synthetic scheme for making embodiments of the current invention.

FIG. 12 shows additional embodiments of the invention.

FIG. 13 shows a synthetic scheme for making embodiments of the current invention.

FIG. 14 shows a synthetic scheme for making embodiments of the current invention.

FIG. 15 shows a synthetic scheme for making embodiments of the current invention.

FIG. 16 show synthetic schemes for making embodiments of the current invention.

FIGS. 17 A and 17B shows a synthetic scheme for making embodiments of the current invention.

FIG. 18 shows a synthetic scheme for making embodiments of the current invention.

FIG. 19 show synthetic schemes for making embodiments of the current invention.

FIGS. 20A and 20B shows a synthetic scheme for making embodiments of the current invention.

FIG. 21 shows a synthetic scheme for making embodiments of the current invention.

FIG. 22 show synthetic schemes for making embodiments of the current invention.

FIGS. 23A and 23B shows a synthetic scheme for making embodiments of the current invention.

FIG. 24 show synthetic schemes for making embodiments of the current invention.

FIGS. 25A and 25B show synthetic schemes for making embodiments of the current invention.

FIG. 26 shows the effect of the experimental MRI tracer, TP-TEMPO on the MRI image of mouse brain measured 48 hours after administration. The image on the left shows an image obtained from a control mouse where no tracer was administered. The image on the right shows the animal treated with TP-TEMPO. The central figure is a chimera directly comparing the left hemisphere of the control with the right hemisphere of the TP-TEMPO treated. In this central image the “Y” shaped region (1) is where the dopaminergic neurons are found.

FIGS. 27-33 illustrate embodiments of the invention wherein R¹⁰ is alkyl or 4-substituted N-methyl tetrahydropyridine.

### DEFINITIONS

The term “antioxidant”, as used herein, refers to any of a variety of substances or chemical compounds that slow or prevent oxidation of compounds. They may inhibit or prevent oxidative degeneration of biological molecules by exposure to compounds including, but not limited to, peroxides, nitroxides, superoxides, or hydroxyl radicals.

The term “compound”, as used herein, refers to any molecule having biological activity and may include, but not limited to, organic molecules, inorganic molecules, proteins, or nucleic acids.

The term “peroxidase activity”, as used herein, refers to any compound that decreases, considering a generally accepted standard deviation, the detection of H₂O₂ generated in the presence of the compound as compared to control (i.e., for example, when lacking the compound). Hydrogen peroxide may be formed by the action of glucose oxidase in a solution containing glucose and detected by the H₂O₂-dependent oxidation of homovanillic acid (3-methoxy-4-hydroxyphenylacetic acid, HVA) to a highly fluorescent dimer (2,2′-dihydroxy-3,3′-dimethoxydiphenyl-5,5′-diacetic acid) mediated by horse-radish peroxidase.

The term “mitochondrial peroxidase activity”, as used herein, refers to any compound capable of reducing mitochondrial hydrogen peroxide production as compared to control (i.e., for example, when lacking the compound) considering a generally accepted standard deviation using appropriately modified procedures as described in Barja J. Bioenerg. Biomembr. 34(3):227-33 (2002) herein incorporated by reference.

The term “neuroprotective of hydrogen peroxide” refers to any compound that decreases caspase-3 activity considering a generally accepted standard deviation compared with control (absent compound) in cellular lysate supernatants. For example, caspase-3 activity may be measured using commercial kits or as previously described. See Kalivendi et al., J. Biol. Chem., Vol. 279, Issue 15, 15240-15247, Apr. 9, 2004.

The term “superoxide dismutase” or “SOD” activity, as used herein, refers to any compound that increases the decomposition of superoxide. Such superoxide decomposition may be measured by using a stopped-flow kinetic analysis as exemplified in Example 13.

The term “conjugate”, as used herein, refers to any compound that has been formed by the joining of two or more moieties. Within the context of certain embodiments, a “moiety” is any type of molecular arrangement designated by formula e.g., chemical name or structure. Within the context of certain embodiments, a conjugate is said to comprise one or more moieties. This means that the formula of the moiety is substituted at some place in order to be joined and be a part of the molecular arrangement of the conjugate. Although moieties may be directly covalently joined, it is not intended that the joining of two or more moieties must be directly to each other. A linking group, i.e., any molecular arrangement that will connect the moieties by covalent bonds such as, but are not limited to, one or more amide group(s), may join the moieties. Additionally, although the conjugate may be unsubstituted, the conjugate may have a variety of additional substituents connected to the linking groups and/or connected to the moieties. For example, a conjugate comprising a first moiety of the formula: 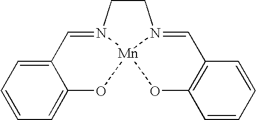
and a second moiety of the formula: 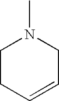
includes a conjugate having the formula: 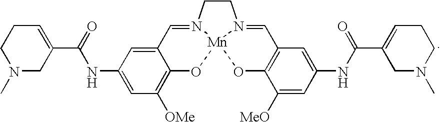
In one embodiment, the amide group acts as a linking group joining the first and second moieties, and the methoxy groups are substituents connected to the first moiety.

The term “aminoxyl radical group” or “nitroxide group” or “nitroxide moiety” or “free radical nitroxide” and the like, as used herein, refers to any compound comprising the molecular arrangement of the radicals structurally designated N—O^{•}, N—O°, or N^{•+-O}⁻. These radicals may be created by removal of the hydrogen atom from the hydroxy group.

The term “hydropyridine moiety”, as used herein, refers to any substituted or unsubstituted heterocyclic molecular arrangement containing all single bonds or one double bond, i.e., tetrahydropyridine, of the following formula:

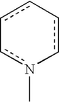

wherein, [symbol] represents a single or double bond. In preferred embodiments, the moiety is a methyl substituted tetrahydropyridine.

The term “tetraalkylheterocyclic nitroxide”, as used herein, refers to any substituted or unsubstituted heterocycle containing a nitrogen of a nitroxide group and four alkyl groups are attached to the carbons in the ring adjacent to the nitrogen, e.g., 2,2,6,6-tetramethyl-piperidine-1-oxyl.

The term “heterocyclic selenium nitroxide”, as used herein, refers to any substituted or unsubstituted heterocycle containing a selenium and a nitrogen of a nitroxide group, e.g., 1,4-diaza-2-selenaccyclohexan-4-oxyl, 1,3-diaza-5-selenacyclohexan-3-oxyl, and 1,3-diaza-6-selenacyclohexan-3-oxyl.

The term “acyl”, as used herein, refers to any substitution comprising an —C(═O)alkyl or —C(═O)aryl group.

The term “adverse drug reaction”, as used herein, refers to any response to a drug that is noxious and unintended that may occur in clinically prescribed doses for prophylaxis, disease diagnosis, or therapy. For example, such reactions may include, but are not limited to, side effects, toxicity, hypersensitivity, drug interactions, complications, or other idiosyncrasies. Side effects are often adverse symptoms produced by a therapeutic serum level of drug produced by its pharmacological effect on unintended organ systems (e.g., for example, blurred vision from anticholinergic antihistamine). A toxic side effect may be an adverse symptom or other effect produced by an excessive or prolonged chemical exposure to a drug (e.g., for example, indomethacin induction of liver toxicity). Hypersensitivities may be immune-mediated adverse reactions (e.g., for example, anaphylaxis, allergy etc.). Drug interactions may comprise adverse effects arising from interactions with other drugs, foods or disease states (e.g., for example, warfarin and erythromycin, cisapride and grapefruit, loperamide and Clostridium difficile colitis). Complications are diseases and/or injury caused by a drug (e.g., aspirin-induced gastric ulcer, estrogen-induced thrombosis). The adverse drug reaction may be mediated by known or unknown mechanisms (e.g., for example, agranulocytosis associated with chloramphenicol or clozapine). Such adverse drug reaction can be determined by subject observation, assay or animal model well-known in the art.

The term “alkyl”, as used herein, means any straight chain or branched, non-cyclic or cyclic, unsaturated or saturated aliphatic hydrocarbon containing from 1 to 10 carbon atoms, while the term “lower alkyl” has the same meaning as alkyl but contains from 1 to 6 carbon atoms. The term “higher alkyl” has the same meaning as alkyl but contains from 2 to 10 carbon atoms. Representative saturated straight chain alkyls include, but are not limited to, methyl, ethyl, n-propyl, n-butyl, n-pentyl, n-hexyl, n-septyl, n-octyl, n-nonyl, and the like; while saturated branched alkyls include, but are not limited to, isopropyl, sec-butyl, isobutyl, tert-butyl, isopentyl, and the like. Cyclic alkyls may be obtained by joining two alkyl groups bound to the same atom or by joining two alkyl groups each bound to adjoining atoms. Representative saturated cyclic alkyls include, but are not limited to, cyclopropyl, cyclobutyl, cyclopentyl, cyclohexyl, and the like; while unsaturated cyclic alkyls include, but are not limited to, cyclopentenyl and cyclohexenyl, and the like. Cyclic alkyls are also referred to herein as a “homocycles” or “homocyclic rings.” Unsaturated alkyls contain at least one double or triple bond between adjacent carbon atoms (referred to as an “alkenyl” or “alkynyl”, respectively). Representative straight chain and branched alkenyls include, but are not limited to, ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include, but are not limited to, acetylenyl, propynyl, 1-butynyl, 2-butynyl, 1-pentynyl, 2-pentynyl, 3-methyl-1-butynyl, and the like.

The term “alkylamino”, as used herein, means at least one alkyl moiety attached through a nitrogen bridge (i.e., —N-(alkyl)_{N}, such as a dialkylamino)) including, but not limited to, methylamino, ethylamino, dimethylamino, diethylamino, and the like.

The term “alkyloxy”, as used herein, means any alkyl moiety attached through an oxygen bridge (i.e., —O-alkyl) such as, but not limited to, methoxy, ethoxy, and the like.

The term “alkylthio”, as used herein, means any alkyl moiety attached through a sulfur bridge (i.e., —S— alkyl) such as, but not limited to, methylthio, ethylthio, and the like.

The term “alkylsulfonyl”, as used herein, means any alkyl moiety attached through a sulfonyl bridge (i.e., —SO2-alkyl) such as, but not limited to, methylsulfonyl, ethylsulfonyl, and the like.

The term “aryl”, as used herein, means any aromatic carbocyclic moiety such as, but not limited to, phenyl or naphthyl.

The term “arylalkyl”, as used herein, means any alkyl having at least one alkyl hydrogen atoms replaced with an aryl moiety, such as benzyl, but not limited to, (CH₂)₂-phenyl, —(CH₂)₃-phenyl, —CH(phenyl)₂, and the like.

The term “derivative”, as used herein, when used in relation to a chemical compound refers to a similar structure that upon application, e.g., administration to a subject, is capable of providing, directly or indirectly, the function said chemical compound is disclosed to have. It is not necessary that the derivative function be identical in effectiveness. For example, the substitution of a hydrogen atom for a methyl group on a compound is a substitution. If the methyl substituted compound has some biological activity that the unsubstituted hydrogen-containing compound was disclosed to have, then the methyl substituted compound is a derivative.

The term “free radical MRI agent”, as used herein, refers to any compound that, when administered to a subject, maybe followed by creating biological images. For example, the interaction of one agent with a magnetic field creates an electron spin resonance signal that is processed by a magnetic resonance spectrometer to provide tissue images of a subject.

The term “halogen”, as used herein, refers to any fluoro, chloro, bromo, or iodo moiety.

The term “haloalkyl”, as used herein, refers to any alkyl having at least one hydrogen atom replaced with halogen, such as trifluoromethyl, and the like.

The term “heteroaryl”, as used herein, refers to any aromatic heterocycle ring of 5- to 10 members and having at least one heteroatom selected from nitrogen, oxygen and sulfur, and containing at least 1 carbon atom, including, but not limited to, both mono- and bicyclic ring systems. Representative heteroaryls include, but are not limited to, furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, or quinazolinyl.

The term “heteroarylalkyl”, as used herein, means any alkyl having at least one alkyl hydrogen atom replaced with a heteroaryl moiety, such as —CH₂pyridinyl, —CH₂pyrimidinyl, and the like.

The term “heterocycle” or “heterocyclic ring”, as used herein, means any 4- to 7-membered monocyclic, or 7- to 10-membered bicyclic, heterocyclic ring which is either saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles may include heteroaryls exemplified by those defined above. Thus, in addition to the heteroaryls listed above, heterocycles may also include, but are not limited to, morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The term “heterocyclealkyl”, as used herein, means any alkyl having at least one alkyl hydrogen atom replaced with a heterocycle, such as —CH₂morpholinyl, and the like.

The term “homocycle” or “homocyclic ring”, as used herein, means any saturated or unsaturated (but not aromatic) carbocyclic ring containing from 3-7 carbon atoms, such as, but not limited to, cyclopropane, cyclobutane, cyclopentane, cyclohexane, cycloheptane, cyclohexene, and the like.

The term “isomers”, as used herein, means any of two or more substances that are composed of the same elements in the same proportions but differ in the three dimensional arrangement of atoms including, but are not limited to, an enantiomeric isomer (i.e., for example, a mirror image) or a diastereomeric isomer (i.e., for example, a non-mirror image).

The term “magnetic resonance spectrometer”, as used herein, means any instrument designed to allow the detection of magnetic resonance including, but not limited to, electron spin resonance (i.e., exciting unpaired electron and/or nuclei and then observing signals as the energy of the particle decays back to the ground state). For example, a magnetic resonance spectrometer may include, but is not limited to, hardware and software for creating images of a mammal from the observed signals (e.g., MRI instruments).

The term “manage”, as used herein, when used in connection with a disease or condition, means to provide any beneficial effect to a subject being administered with a prophylactic or therapeutic agent that may not necessarily result in a cure of the disease. In certain embodiments, a subject is administered with one or more prophylactic or therapeutic agents to manage a disease so as to prevent the progression or worsening of the disease.

The term “salts”, as used herein, refers to any salt that complexes with identified compounds contained herein while retaining a desired biological activity. Examples of such salts include, but are not limited to, acid addition salts formed with inorganic acids (e.g. hydrochloric acid, hydrobromic acid, sulfuric acid, phosphoric acid, nitric acid, and the like), and salts formed with organic acids such as, but not limited to, acetic acid, oxalic acid, tartaric acid, succinic acid, malic acid, fumaric acid, maleic acid, ascorbic acid, benzoic acid, tannic acid, pamoic acid, alginic acid, polyglutamic, acid, naphthalene sulfonic acid, naphthalene disulfonic acid, and polygalacturonic acid. Salt compounds can also be administered as pharmaceutically acceptable quaternary salts known by a person skilled in the art, which specifically include the quaternary ammonium salts of the formula —NR, R′, R″⁺Z⁻, wherein R, R′, R″ is independently hydrogen, alkyl, or benzyl, and Z is a counter ion, including, but not limited to, chloride, bromide, iodide, alkoxide, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate). Salt compounds can also be administered as pharmaceutically acceptable pyridine cation salts having a substituted or unsubstituted partial formula: 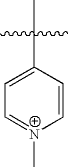
Z⁻, wherein Z is a counter ion, including, but not limited to, chloride, bromide, iodide, alkoxide, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate).

As used herein, the terms “prevent” and “preventing” refer to the slowing and/or delay of the recurrence, spread, and/or onset of a disease or other detrimental biological condition. It is not intended that the term refer to complete prevention. Delayed onset, or a reduction in disease symptom severity is also within the scope of this term.

The term, “impurities”, as used herein, refers to any unwanted reaction products that are not isomers formed during synthesis and does not include residual solvents remaining from the process used in the preparation of the composition or excipients used in pharmaceutical preparations. For example, a purified composition made in accordance with the invention preferably contains less than 10% mass/mass (m/m), but more preferably less than 3% m/m, of “impurities”.

The term “essentially free”, as used herein, when referring to a specific molecule, means that the specific molecule is present in a composition only as an unavoidable impurity. Preferably a composition has less than 0.5%, 1.0%, 3%, 5%, 8% of impurities.

The term “subject”, as used herein, means any animal, including but not limited to a human, domestic livestock (i.e., for example, cattle, sheep, horses etc), domestic pets (i.e., for example, dogs, cats etc), and non-domesticated animals (i.e., for example, lions, tigers, elephants etc.). A human “subject” may also refer to a patient under the care of medical personnel, either on an in-patient or outpatient basis.

The term “substituted”, as used herein, means at least one hydrogen atom of a molecular arrangement is replaced with a substituent. In the case of an oxo substituent (“═O”), two hydrogen atoms are replaced. When substituted, one or more of the groups below are “substituents.” Substituents include, but are not limited to, halogen, hydroxy, oxo, cyano, nitro, amino, alkylamino, dialkylamino, alkyl, alkoxy, alkylthio, haloalkyl, aryl, arylalkyl, heteroaryl, heteroarylalkyl, heterocycle, and heterocyclealkyl, as well as, —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. In addition, the above substituents may be further substituted with one or more of the above substituents, such that the substituent comprises a substituted alky, substituted aryl, substituted arylalkyl, substituted heterocycle, or substituted heterocyclealkyl. Rₐ and R_{b} in this context may be the same or different and, independently, hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl.

The term “unsubstituted”, as used herein, refers to any compound does not contain extra substituents attached to the compound. An unsubstituted compound refers to the chemical makeup of the compound without extra substituents, e.g., the compound does not contain protecting group(s). For example, unsubstituted proline is a proline amino acid even though the amino group of proline may be considered disubstituted with alkyl groups.

The terms “treat” and “treating”, as used herein, refers to any administration of a compound to a subject for the purpose of clinical therapy. The term is not limited to the case where the subject (e.g. for example, a patient) is cured and the disease is eradicated. Rather, the term also contemplates treatment that merely reduces symptoms, and/or delays disease progression.

### DETAILED DESCRIPTION OF THE INVENTION

The invention relates to compounds and compositions that function as antioxidants to treat some neurodegenerative diseases. In some embodiments, the invention relates to an antioxidant comprising a selenium atom and nitroxide group. In further embodiments, the antioxidant comprises peroxidase and superoxide dismutase activity. In some embodiments, the antioxidants are effective in treating neurodegenerative diseases including, but not limited to, Alzheimer's disease, Parkinson's disease, or multiple sclerosis. In additional embodiments, the invention relates to using compounds disclosed herein as free radical electromagnetic imaging agents.

I. Intramitochondrial Dopamine Transporters

Dopaminergic neurons of the midbrain are the main source of dopamine (DA) in the mammalian central nervous system. Dopaminergic neurons are found in a ‘harsh’ region of the brain, the substantia nigra pars compacta, which is DA-rich and contains both redox available neuromelanin and a high iron content. Although their numbers are few, these dopaminergic neurons play an important role in the control of multiple brain functions including voluntary movement and a broad array of behavioral processes such as mood, reward, addiction, and stress.

Mitochondria are known to accumulate reactive oxygen and nitrogen species because mitochondria have a negative membrane potential (i.e., by maintaining a negatively charged inner membrane) and, consequently, tend to accumulate lipophilic cations. Alternatively, compounds may accumulate within the mitochondria by specific active transport systems. One such transport system comprises dopamine transport into the mitochondria.

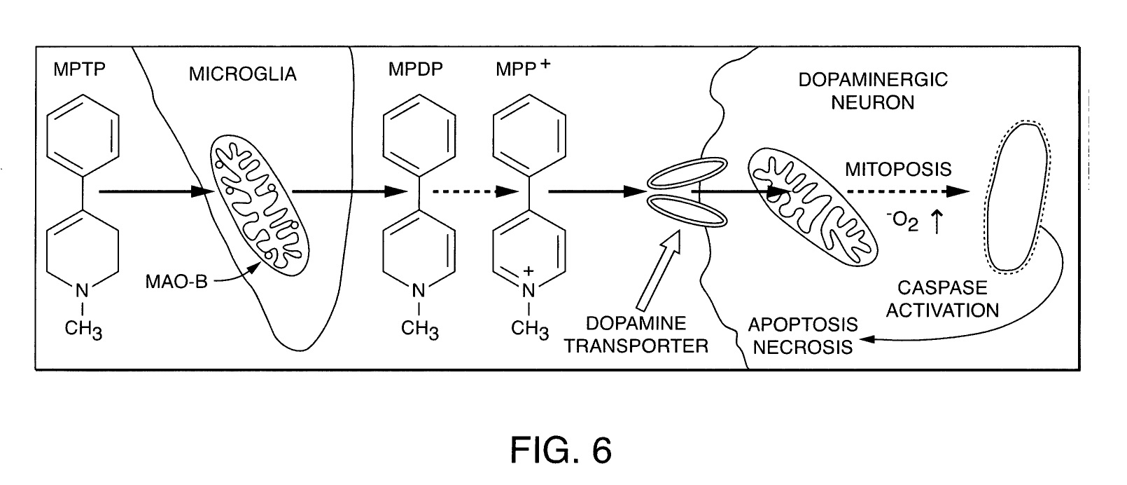

The dopamine transporter system also carries compounds such as, but not limited to, N-Methyl-4-phenyl-1,2,3,6-tetrahydropyridine (MPTP). MPTP is reported to be a neurotoxin specific to the dopaminergic neurons of the sustantia nigra and used in animal models of Parkinson's disease. MPTP is believed able to cross the blood/brain barrier. See FIG. 6. Once inside the brain, MPTP can diffuse within the extracellular space until reaching a microglial monamine oxidase-B (MAO-B) where it is oxidized to N-Methyl-4-phenylpyridine cation (MPP⁺) via oxidation through the intermediate 1-Methyl-4-phenyl-2,3-dihydropyridinium (MPDP). MPDP or MPP⁺ exit the microglia into the intracellular space until provided access into a dopaminergic neuron by a plasmalemmal dopamine transporter protein. This particular active transport of pyridinium cation is unique to dopaminergic neurons. Typically cations do not interfere with the dopamine transporter because cations do not usually cross the blood/brain barrier. However, generation of MPP⁺ via MAO-B bypasses this mechanism. Once inside the dopaminergic neuron, MPP⁺ binds to a mitochondrial dopamine transporter protein and accumulates within a mitochondrion. One activity of MPP⁺ is believed to inhibit mitochondrial complex I thereby causing generation of superoxide ions. Inhibition of mitochondrial complex I may thereby cause: i) an overall cellular energy deficit; ii) decreased the production of APT and glutathione; and iii) increased production of reactive oxygen species. The cellular energy deficit and release of cytochrome c also leads to apoptosis/necrosis and glial activation.

In some embodiments, the invention relates to a compound that can function as a catalytic antioxidant capable of specifically targeting the mitochondria of dopaminergic neurons. Although it is not intended that the invention be limited by any particular mechanism, it is believed that once a compound enters the mitochondria, cell survival is aided by the deactivation of deleterious reactive oxygen and nitrogen moieties.

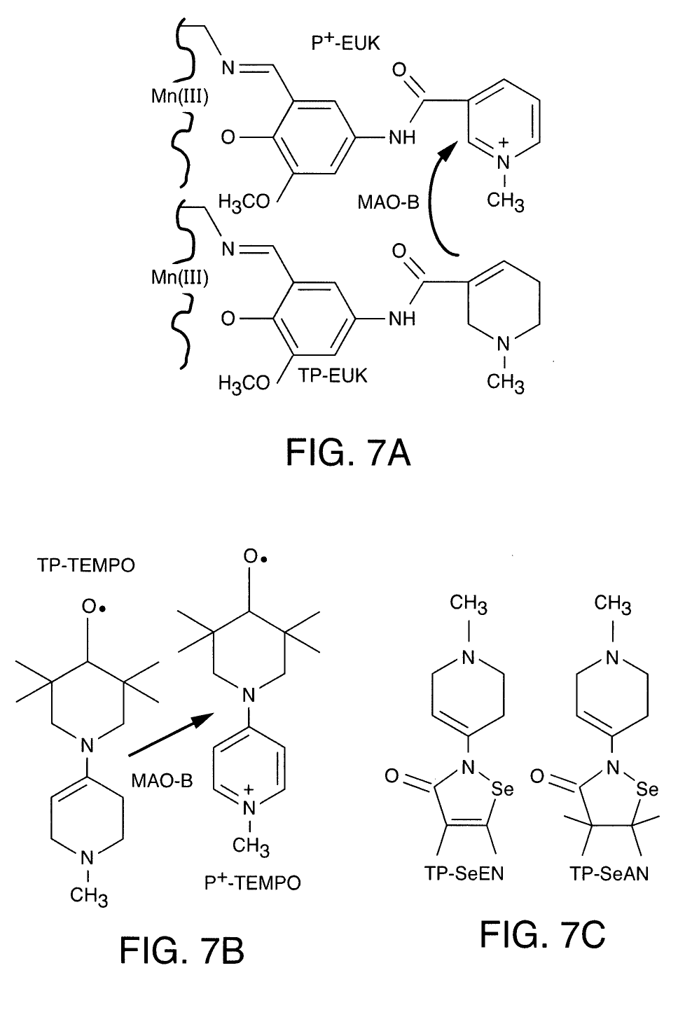

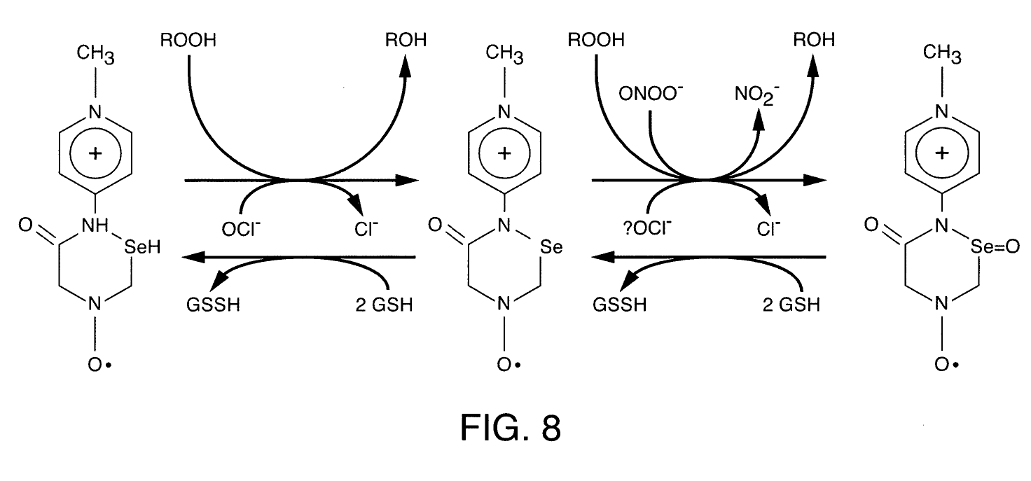

In some embodiments, the invention relates to selenium containing compounds comprising a hydropyridine group. See FIG. 7 and FIG. 8. Although it is not intended that the invention be limited by any particular mechanism, it is believed that the hydropyridine compounds are preferentially accumulated within the mitochondria. For example, the hydropyridine containing compound MPTP (e.g., 1-methyl-4-phenyl-1,2,5,6-tetrahydropyridine) has dopaminergic specificity because the compound crosses the blood brain barrier as a neutral species, and it is converted to the pyridinium cation by gial cells via monamine oxidase. Subsequently, the pyridinium cations may be taken up by dopaminergic neuron via a first dopamine transporter, and once in the cells, the cation is available for accumulation within the mitochondria by a second dopamine transporter (i.e., for example, a trans-mitochondrial membrane dopamine transporter). Pyridinium compounds can catalytically deactivate reactive oxygen/nitrogen species (e.g., free radicals) present in the mitochondria thereby improving dopaminergic cell viability and promote recovery from cellular damage and return to health. In some embodiments, pyridinium compounds increase dopamine levels and allow the cells to survive during high levels of L-DOPA induced stress.

II. Physiological Oxidation/Reduction Mechanisms

In some embodiments, the present invention contemplates providing antioxidant agents that are capable of detoxifying superoxide, hydrogen peroxide, and hydroxy radicals. Another embodiment contemplates providing antioxidant agents that are capable of detoxifying most, if not all, biologically significant reactive oxygen and reactive nitrogen species. Other desirable properties that these antioxidant agents have include, but are not limited to: i) solubility characteristics that allow an agent to have sufficient bioavailability when administered as a therapeutic formulation, ii) low general cellular toxicity, including any metabolites, iii) solubility in both membrane and aqueous cellular phases thereby allowing an agent to cross the blood brain barrier and/or detoxify lipoperoxides; and iv) susceptible to chemical modification in order to attach an agent to a targeting moiety thereby forming a conjugate that can target an agent to a particular organ, tissue, and/or cell type.

A. Reactive Oxidation Species

Biologically reactive oxygen species include, but are not limited to: i) superoxide (O₂⁻); ii) peroxides (ROOH) such as, but not limited to, hydrogen peroxide (H₂O₂) or hypochlorite (OCl⁻); and iii) hydroxide radical (OH). Biologically reactive nitrogen species include, but are not limited to, nitric oxide (NO), nitrogen dioxide (NO₂), or peroxynitrate (ONOO⁻).

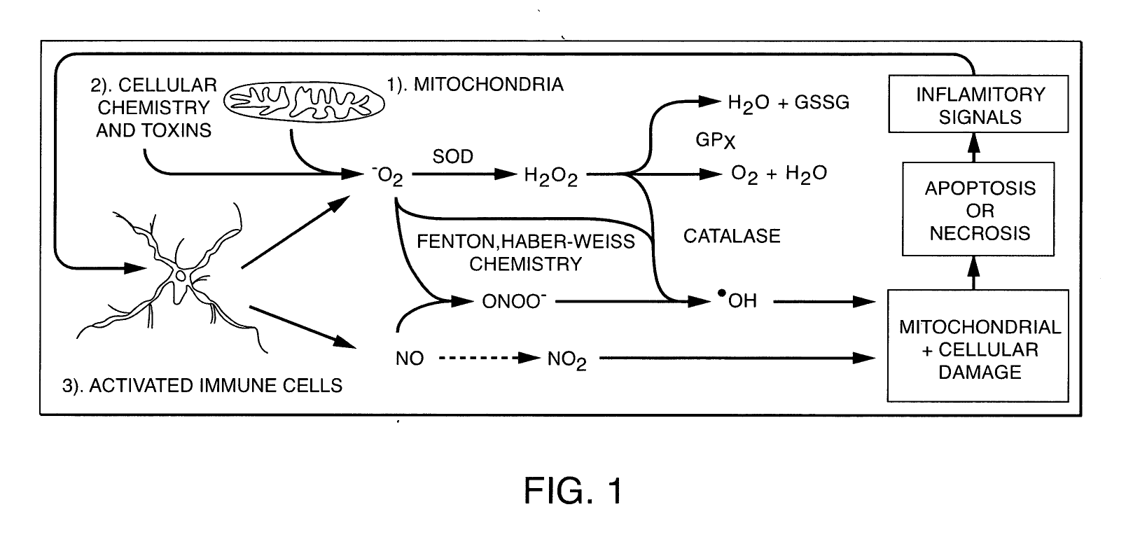

Any interplay of these various reactive species may play a role in the development and progression of neurodegenerative diseases including, but not limited to, Parkinson's disease. Generally, it is believed that these reactive species, particularly the free radical derivatives, react with, breakdown, and interrupt normal biomolecular mechanisms. As depicted in FIG. 1, a superoxide radical is generated from molecular oxygen by; i) NADH oxidase/NO synthase in activated microglia; ii) autoxidation of metabolites such as dopamine; and iii) leakage of electrons from the mitochondrial respiratory chain. Further, superoxide and nitric oxide may interact to generate peroxynitrate. Hydrogen peroxide and peroxynitrate may then further interact to generate a hydroxide radical. Hydroxide radicals are believed a particularly damaging oxidant because it initiates a free radical cascade pathway.

The traditional “free radical theory of aging” implicates an increase in the steady-state levels of damaging biomolecules due to reactive species including superoxide, hydrogen peroxide, and hydroxyl radicals. Mammals have specific enzymes to dismutate (i.e., for example, reduce) superoxide into oxygen and hydrogen peroxide such as, but not limited to: i) cytosolic Cu/Zn enzymes; ii) extracellular Cu/Zn enzymes, and iii) mitochondrial Mn-containing enzymes. Alternatively, hydrogen peroxide can be broken down into water and oxygen by catalase or glutathione peroxidase. It is believed that life expectancy may decline with an increasing generation of superoxide and hydrogen peroxide in many species and in organisms with low levels of superoxide dismutases and catalase. For example, it has been observed that the life expectancy of Drosophila can be extended up to one-third in mutants that overexpress both superoxide dismutases (SODs) and catalase.

B. Manganese-Salens Antioxidants

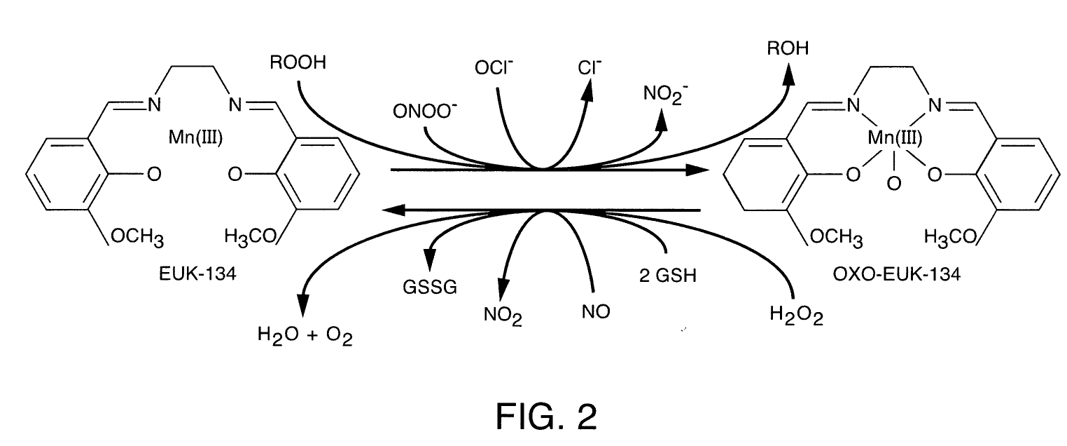

In some embodiments, the present invention contemplates a catalytic antioxidant that functions to deactivate or detoxify reactive oxygen species and/or reactive nitrogen species induced cellular stress. In one embodiment, compounds referred to as Manganese Salens or Mn-Salens comprise manganese complexes of substituted or unsubstituted 2-[2-[(2-hydroxyphenyl)methylideneamino]ethyliminomethyl]phenol and have catalase and peroxidase activity. In one embodiment, Mn-salen compounds detoxify a range of oxygen donors by generating oxo-Mn-salen compounds, which are themselves less reactive than reactive oxygen species and/or reactive nitrogen species. See FIG. 2. In another embodiment, the oxo-Mn-salen can react with thiols, including glutathione (GSH), to form disulfides.

Although it is not necessary to understand the mechanism of an invention, it is believed that Mn-salens may act as a superoxide dismutase, wherein the Mn(III) group of Mn-salens is redox active and is reduced to Mn(II) by superoxide, which can further react with another molecule of superoxide to form hydrogen peroxide and Mn(III). It is further believed that manganese antioxidants are desirable because the produced hydrogen peroxide is not further reduced to form a hydroxide radical; a very strong reactive oxygen species. Hydroxide radicals usually form in the presence of hydrogen peroxide and redox active metals, e.g., in Fenton's reagent hydrogen peroxide and an iron form a solution of hydroxide radicals typically used to oxidize contaminants in wastewaters.

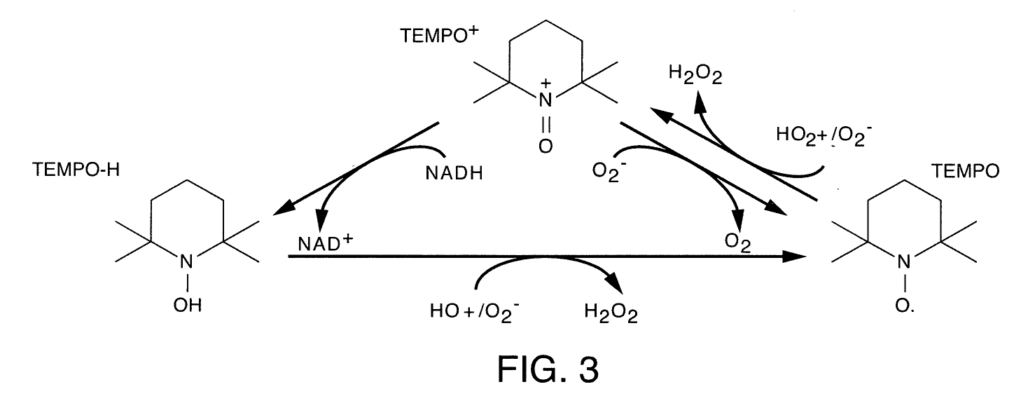

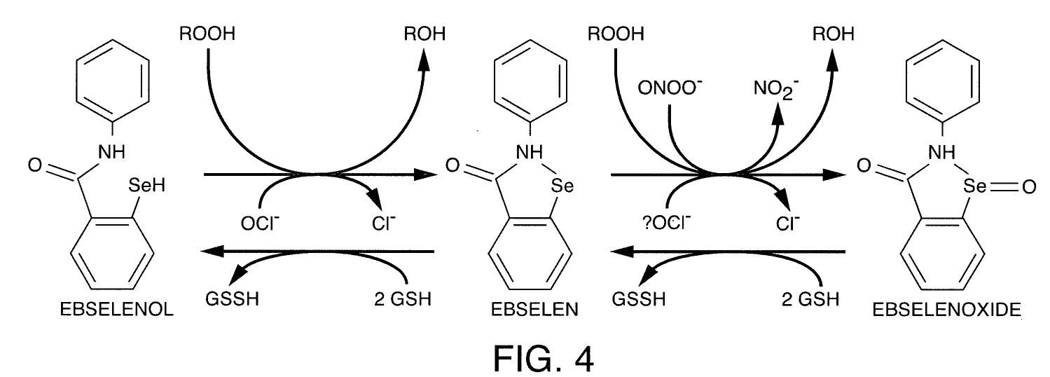

In one embodiment, Mn-salen antioxidant conjugates comprise artificial super oxide dismutase/catalase mimetics. In one embodiment, a Mn-salen antioxidant conjugate may include chemical moieties selected from the group comprising nitroxides (i.e., for example, 2,2,6,6-tetramethyl-1-piperidinyloxy (TEMPO): see FIG. 3); and selenides (i.e., for example, Ebselen: see FIG. 4). In one embodiment, Mn-salen conjugates ameliorate oxidative and/or nitrosative stresses. Mn-salens promote cell viability and tissue function for a number of disease models including, but not limited to, stroke, Amyotrophic lateral sclerosis, multiple sclerosis, excitotoxic neuronal injury, ischemia/reperfusion injury in heart and kidney tissue, organ failure in endotoxic and hemorrhagic shock, Alzheimer's or Parkinson's disease.

C. Selens Antioxidants

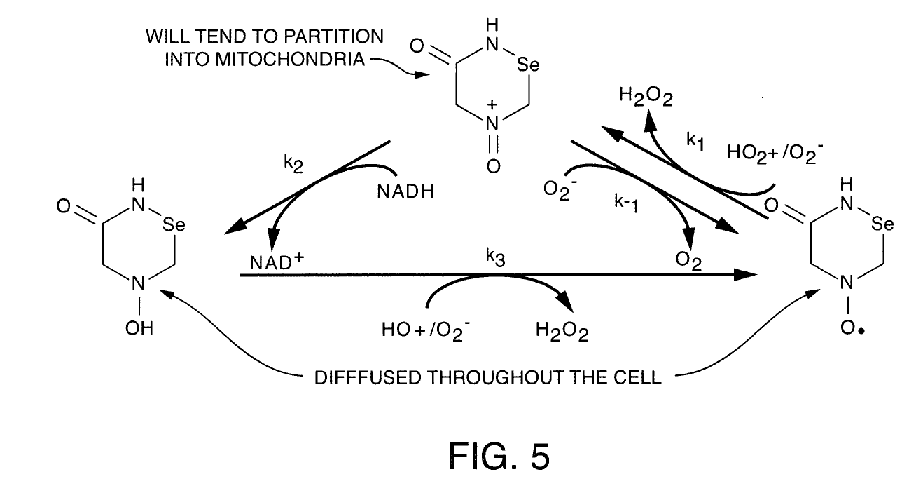

In some embodiments, the invention contemplates a compound comprising selenium and heterocyclic nitroxides that function as multifunctional antioxidants. (i.e., for example, SeleNO1; 1,4-diaza-2-selencyclohex-6-one-4-oxyl). See FIG. 5. Although it is not intended that the invention be limited to any particular mechanism, it is believed that SeleNO1 is a bifunctional antioxidant because it contains both selenide (—Se—) and nitroxide (—NO—) groups. A selenide moiety may confer peroxidase activity within a subject, whereas a nitroxide moiety may confer superoxide dismutase activity. In one embodiment, a bifunctional antioxidant reduces mitochondrial permeability transition by arresting cytochrome c induced apoptosis that occurs in neurodegenerative diseases.

It is further believed that SeleNO1 can be oxidized to the oxy-ammonium cation SeleNO1⁺ by peroxy radicals and superoxide. SeleNO1⁺ can be reduced back to SeleNO1 by superoxide. Alternatively, SeleNO1⁺ can be reduced back to SeleNO1 by a hydride transferring reductant such as NADH, to the hydroxylamine, SeleNO—H. Although it is not necessary to understand the mechanism of the invention, it is believed that the structure of SeleNO1 is such that it is quite lipophilic in character, and therefore may cross biological membranes and preferentially partition into mitochondria.

The antioxidant, Ebselen (e.g., 8-phenyl-7-selena-8-azabicyclo[4.3.0]nona-1,3,5-trien-9-one) is able to react with oxygen donating oxidants to form a selenoxide which can be reduced back to Ebselen by glutathione or thioredoxin. See FIG. 4. A selens moiety may also be reduced directly to selenols, e.g., N-phenyl-2-selanyl-benzamide, by glutathione or thioredoxin, and selenols are per-species scavengers. Glutathione is a cysteine containing tripeptide with many roles in cells and is involved in protein disulfide bond rearrangement and reduces peroxides. Glutathione also is known to regulate enzyme activity in the formation of disulfide bonds through S-glutathiolation. These derivatives are preferred because the hydrophobic benzene ring in Ebselen imparts poor water solubility, a property that is not desirable for a therapeutic agent. In addition, Ebselen has relatively poor glutathione peroxidase-like catalytic activity because of thiol exchange reactions that take place at the selenium center.

D. Nitroxide Antioxidants

One limitation of Ebselen relates to the detoxification of oxidative radicals such as superoxide. Nitroxides, however, can detoxify superoxide at levels found in brain pathophysiology. Nitroxides may comprise free radicals that function as superoxide dismutases. In one embodiment, a moiety comprising 2,2,6,6-tetramethyl-1-piperidinyloxy (e.g., TEMPO) detoxifies superoxide. Although it is not necessary to understand the mechanism of an invention, it is believed that TEMPO detoxifies superoxide by cycling between the nitroxide, hydroxylamine, and an amine cation. See FIG. 3. It is further believed that in the presence of a superoxide and a peroxy radical, TEMPO is oxidized to the oxy-ammonium cation (e.g., TEMPO⁺) thereby forming hydrogen peroxide. TEMPO⁺ can then be reduced by another molecule of superoxide back to TEMPO. Alternatively, TEMPO⁺ can then also be further reduced by another a hydride transferring reductant such as NADH, to a hydroxylamine (e.g., TEMPO-H) which is also a substrate for superoxide and hydroxy radical to form hydrogen peroxide. Nitroxides do not typically react with peroxides such as hydrogen peroxide that Ebselen is able to decompose to oxygen and water.

III. Neurodegenerative Diseases

Neurodegenerative diseases include, but are not limited to, Alzheimer's disease, Parkinson's disease, multiple sclerosis, amyotrophic lateral sclerosis (ALS or Lou Gehrig's Disease), Huntington's disease, frontotemporal dementia (Pick's Disease), and prion diseases (i.e., for example, ‘mad cow’ disease).

A. Alzheimer's Disease

The anatomic pathologies of Alzheimer's disease (AD) include, but are not limited to, i) neurofibrillary tangles (NFTs); ii) microscopic senile plaques (SPs); and iii) cerebrocortical atrophy. These pathologies have been suggested to be predominantly involved with the association regions and, in particular, the medial aspect of the temporal lobe. Although neurofibrillary tangles and senile plaques are characteristic of Alzheimer's disease, they are not pathognomonic because other neurodegenerative conditions distinct from Alzheimer's disease are characterized by neurofibrillary tangles (e.g., progressive supranuclear palsy, dementia pugilistica) or senile plaques (e.g., normal aging). Other lesions of AD include, but are not limited to: i) granulovacuolar degeneration; ii) neuropil threads; and iii) neuronal loss and/or synaptic degeneration, which are thought to ultimately mediate the cognitive and behavioral manifestations of the disorder.

To date, no interventions have been shown to convincingly prevent Alzheimer's disease or slow its progression. Medical treatments for Alzheimer's disease may include, but are not limited to, psychotropic medications or behavioral interventions. Medications that many practitioners prefer include, but are not limited to, haloperidol, risperidone, olanzapine, or quetiapine. Adverse reactions to conventional neuroleptics have motivated those in the art to search for new agents that can alleviate disruptive behavior while minimizing the occurrence of extrapyramidal manifestations and/or a worsening of motor and behavioral performance. Some Alzheimer's disease patients are given high doses of tocopherol (2,5,7,8-tetramethyl-2-(4,8,12-trimethyltridecyl)chroman-6-ol) that has antioxidant activity by virtue of a phenolic hydrogen on the 2H-1-benzopyran-6-ol nucleus. However, high-doses of tocopherol may sometimes cause adverse cardiovascular events.

B. Multiple Sclerosis

Multiple sclerosis (MS) is believed to result from damage to myelin—a protective sheath surrounding nerve fibers of the central nervous system. Myelin damage is further believed to interfere with neuronal messages (i.e., for example, action potential trafficking) relayed between the brain and other parts of the body. MS symptoms include, but are not limited to, blurred vision, weak limbs, tingling sensations, unsteadiness and fatigue. The most common symptoms of MS comprise a multicentric and/or multiphasic central nervous system inflammation and demyelination. Further, lesion formations characteristically involve the optic nerve and periventricular white matter of the cerebellum, brain stem, basal ganglia, and spinal cord. MS may further be characterized by periods of relapse and remission, but some cases have a steady progressive pattern.

The diagnosis of MS is typically based on a classic symptom presentation including, but not limited to, optic neuritis, transverse myelitis, internuclear opthalmoplegia, paresthesias, and other neurologic abnormalities. About 70% of patients present with a more favorable relapsing-remitting (RR) type, which is characterized by acute exacerbations with full or partial remissions. For patients with RR, the FDA has approved the long-term use of beta-interferons and glatiramer acetate, which is a synthetic form of myelin basic protein (MBP) that has fewer side effects than interferon. Both interferon and glatiramer treatments have demonstrated reductions in both clinical disease activity and progression of multiple sclerosis lesions. Chronic progressive multiple sclerosis, which many patients with RR develop over time, is subdivided further into: i) primary-progressive (PP); ii) relapsing-progressive (RP) having characteristics of both RR and RP and elicits intermediate clinical severity; and iii) secondary-progressive (SP).

Multiple sclerosis commonly is believed to primarily result from an autoimmune process. A non-random geographic distribution, however, suggests that secondary causes including, but not limited to, an environmental effect, or an inadvertent activation and/or dysregulation of central nervous system's immune processes may also play a role. Pharmacological treatments of progressive MS or relapse prevention may include, but are not limited to, of interferon, cyclosporine, azathioprine, methotrexate, or other immunomodulatory agents.

C. Amyotrophic Lateral Sclerosis

Amyotrophic lateral sclerosis, often referred to as “Lou Gehrig's disease,” is a progressive neurodegenerative disease that affects nerve cells in the brain and the spinal cord. No cure or effective treatment presently exists. Motor neurons reach from the brain to the spinal cord and from the spinal cord to the muscles throughout the body. The progressive degeneration of the motor neurons in amyotrophic lateral sclerosis eventually lead to their death. When the motor neurons die, the ability of the brain to initiate and control muscle movement is lost. With voluntary muscle action progressively affected, patients in the later stages of the disease may become totally paralyzed. Amyotrophic lateral sclerosis primarily involves anterior horn cells in the spinal cord and cranial motor nerves.

Patient symptomologies may include, but are not limited to, weakness of bulbar muscles or of single or multiple limb muscle groups. Such muscle weakness is not, however, always bilateral or symmetrical. A predominantly bulbar form usually leads to more rapid deterioration and death. Limb weakness is predominantly distal. Weakness and atrophy of the intrinsic hand muscles are prominent. Weakness progresses to involve the forearms and shoulder girdle muscles and the lower extremities.

The discovery that a small percentage of ALS cases are familial and involve mutation in a superoxide dismutase gene (SOD1) led to the development of transgenic mouse models presently widely used for testing possible drugs. No breakthrough has yet occurred and those in the art currently believe that combinations of drugs may be required to slow the multifactorial neurodegeneration process effectively.

Many different types of drugs have been tested; most are based on various hypotheses of mechanisms for neuronal death, including oxidative damage, loss of trophic factor support, glutamate-mediated excitotoxicity, and chronic inflammation. Riluzole (6-(trifluoromethoxy)-benzothiazol-2-amine), an inhibitor of glutamate release, is the only agent presently approved for clinical use but extends survival by a few months.

D. Parkinson's Disease

Parkinson's disease is a gradual progressive central neurodegenerative disorder that affects body movement and is characterized by symptoms including, but not limited to, muscle rigidity, resting tremors, loss of facial expression, hypophonia, diminished blinking, or akinesia. The major neuropathologic findings in Parkinson's disease are a loss of pigmented dopaminergic neurons in the substantia nigra and the presence of Lewy bodies. Lewy bodies are concentric, eosinophilic, cytoplasmic inclusions with peripheral halos and dense cores. The presence of Lewy bodies within pigmented neurons of the substantia nigra is characteristic of Parkinson's disease; however, they are also found in the cortex, nucleus basalis, locus ceruleus, intermediolateral column of the spinal cord, and other areas.

The motor disabilities characterizing Parkinson's disease are primarily due to the loss of dopaminergic neurons in the substantia nigra resulting in a dramatic decrease in the dopamine levels in the brain. The generation of dopamine requires the expenditure of energy and this energy is normally provided by neuronal mitochondria. Mitochondria are believed to be organelles that generate energy within most of our cells, and their function plays a role in cell function and survival. Once dopaminergic neuronal cell death reaches the critical level of 85-90%, the neurological symptoms of Parkinson's disease appear. The original treatment for Parkinson's disease was the systematic administration of levodopa (L-DOPA), a precursor to dopamine that enters the brain via a carrier-mediated transport system where it is converted to dopamine by the enzyme L-aromatic amino acid decarboxylase (L-AAAD). Striatal dopamine is deficient in Parkinson's disease and its replacement with high dosages of L-DOPA is thought to ameliorate the symptoms of the disease. Several problems usually develop during the chronic use of L-DOPA. The most common and vexing problems are dyskinesias. Further, most pharmacological treatments become compromised by the development of functional tolerance and the therapy ultimately becomes ineffective. As oxidative damage and mitochondrial dysfunction are a major cause of dopaminergic cell death, Parkinson's disease and other neurological disorders are possibly exacerbated by L-DOPA induced oxidative stress.

Selegiline (N-methyl-1-phenyl-N-prop-2-ynyl-propan-2-amine) is an irreversible inhibitor of type B monoamine oxidase (MAO-B) and is usually administered to newly diagnosed patients with Parkinson's disease because it slows the disease's progression. However, it is believed that selegiline also affords a neuroprotective effect for dopamine neurons independent of MAO-B inhibition because it has been found that selegiline induces transcriptional events that result in increased synthesis of antioxidants and anti-apoptotic proteins. Recent evidence indicates that one of selegiline's metabolites, desmethylselegiline, is the active agent for neuroprotection.

Postmortem tissue studies of Parkinson's disease patients show evidence of: i) increased chemical levels of lipid peroxidation, protein oxidation, 3-nitrotyrosine formation, DNA oxidation and breaks; and ii) a decrease in the activities of the reactive oxygen species (ROS) scavenging enzymes glutathione peroxidase (GPx), and superoxide dismutase (SOD). See FIG. 1. This evidence suggests that Parkinson's disease may involve mitochondrial oxidative stress.

One model of mitochondrial oxidative stress may occur by exposure to 1-methyl-4-phenylpyridinium ions (MPP⁺) which is the active metabolite of 1-methyl-4-phenyl-1,2,3,6-tetrahydropyridine (MPTP). See FIG. 6. Rotenone, MMP+, and 6-hydroxydopamine (6-OHDA) all induce a Parkinsonian-like syndrome in primates and rodents. These neurotoxins are believed to inhibit mitochondrial complex I, and mimic the pathology of Parkinson's disease in animal research models.

Reactive oxygen and nitrogen species can also cause programmed cell death (i.e., apoptosis) in many cell types mediated by a mitochondrial cytochrome c activated caspase 9 and/or caspase 3 pathway (i.e., for example, the caspase 9/3 cascade). It has been observed that neuronal mitochondria release cytochrome c following rotenone exposure in a dose dependent manner in either isolated mitochondria or intact primary neurons assay techniques. Rotenone induced death can be arrested by treatment with inhibitors of the caspase 9/3 cascade. Postmortem studies indicate that dopaminergic neuron loss in Parkinson's disease is due, at least in part, to the cytochrome c dependant, caspase-9/3 initiated apoptotic pathway. In some embodiments, the present invention contemplates a compound that is neuroprotective because it protects mitochondrial viability and prevents apoptotic cascade mechanisms.

Neuropathological studies show about a 30% defect in mitochondrial complex I function in deceased Parkinson's disease patients, as compared with aged matched controls. Dopaminergic neurons have been shown to have mitochondria with low activity of complex I, and thus resemble rotenone treated mitochondria. Thus, it is believed that mitochondrial damage (i.e., for example, oxidative stress) may be a cause of dopaminergic neuronal death. It is not intended that the invention be limited to any particular mechanism, but it is believed that dopaminergic neuronal death can occur for reasons including, but not limited to: i) dopaminergic neuron mitochondria may be selectively vulnerable to some environmental contaminants that cause mitochondrial dysfunction; ii) dopaminergic neurons may produce an inherent mitochondrial toxin; or iii) mitochondria harbor endogenous defects in enzymes such as complex I that lead to impaired energy metabolism.

In some embodiments, the invention relates to targeting the mitochondria of dopaminergic neurons with nitroxide compounds disclosed herein for diagnosis of Parkinson's disease. Parkinson's disease is characterized by a deficit in dopamine, a neurotransmitter produced in a part of the brain called the substantia nigra (SN). The SN is divided into 2 components that have different connections and distinct neurotransmitters; a more ventral part called the substantia nigra pars reticulata (SNr) and a dorsal part the substantia nigra pars compacta (SNc). Dopamine is responsible for maintaining normal motor functioning and is produced by specialist dopaminergic neurons in the SNc. The SN is a very small, though vital, part of the brain. The mean (+/−SD) thickness values of the substantia nigra were 5.1+/−0.89 mm in control subjects, 4.8+/−0.75 mm in patients with Parkinson's disease, and 3.4+/−0.53 mm in patients with secondary Parkinsonism. Although more advanced techniques, such as fractional anisotropy MRI are able to show small changes in the size of the SNc, in advanced PD patients, it is not as present possible to track the development of PD from its early symptomatic stages using MRI. Thus there is a need to magnetically contrast the dopaminergic neurons of the SN against the background of the brain.

It is also believed that inflammation contributes to the pathogenesis of Parkinson's disease. The sustantia nigra comprises a layer of large pigmented nerve cells in the midbrain that produce dopamine and whose destruction is associated with Parkinson's disease. Microglial cells (i.e., for example, a specialized neuroglial cell) are dispersed within the substantia nigra comprising phagocytic cells that are active in immune reactions of the central nervous system. When activated, neuroglia generate large amounts of reactive nitrogen and oxygen species. Microglia activation markers have been observed in, and localized to, the substantia nigra in Parkinson's disease.

Mitochondrial toxin exposure causes extensive microglial activation in the rat striatum and nigra, with less in the cortex, and microglial morphology in striatum of rotenone-treated animals resembles that seen in Parkinson's patients. No differences were observed between controls and autopsied Parkinson's patients in terms of distribution, cellular density, or cellular morphology of astrocytes in the substantia nigra. A similar observation was made in a number of rodent Parkinson's disease models induced by mitochondrial toxins. Microglial activation occurs in the nigrostriatal pathway in both MPTP and 6-OHDA treated Parkinson's disease models, and anti-inflammatory agents that inhibit activation of microglia (e.g. minocycline) attenuated both MPTP and 6-OHDA toxicity. Thus, one embodiment of the present invention contemplates preventing or attenuating the effects of microglia activation by detoxifying both reactive oxygen species and reactive nitrogen species thereby relieving intramitochondrial oxidative stress.

E. Frontotemporal Dementia

Frontotemporal dementia (FTD) comprises a focal clinical syndrome characterized by profound changes in personality and social conduct and associated with a circumscribed degeneration of the prefrontal and anterior temporal cortex. Onset is typically in the middle years of life and survival is about 8 years. The presence of microtubule-associated-protein-tau-based pathological features in some patients and the discovery, in some familial cases, of mutations in the tau gene links FTD to other forms of tauopathy, such as progressive supranuclear palsy and corticobasal degeneration. However, more than half of all patients with FTD, including some with a strong family history, show no apparent abnormality in the tau gene or protein, indicating pathological and etiological heterogeneity. FTD provides a challenge both for clinical management and for theoretical understanding of its neurobiological substrate.

The initial symptoms typically occur without affecting other cognitive domains, such as memory, and rarely present with an onset age beyond 75 years. In some instances, deficits in behavior and language are also accompanied by Parkinsonism or progressive motor neuron disease. While 25-40% of all FTD cases are believed to be familial, the clinical and neuropathological variability of the syndrome suggests the existence of several distinct genetic factors underlying or modifying pathogenesis.

The onset is usually slow and insidious. The disorder involves shrinking of the tissues (atrophy) of the frontal and temporal lobes of the brain, “fronto-temporal dementia.” The neurons (nerve cells) in the affected areas contain abnormal material (Pick's bodies). These are tangles made of tau protein. The exact cause is unknown.

The symptoms may be similar to Alzheimer's, with aphasia (loss of language abilities), agnosia (loss of ability to recognize objects or people), and apraxia (loss of skilled movement abilities). Behavioral changes are prominent with loss of inhibition and change in personality, as opposed to Alzheimer's disease, where memory loss is often the primary feature. Some personality changes include, but are not limited to, striking loss of concern and lack of anxiety, loss of initiative, flat affect (does not display any emotion), indecision, inappropriate mood, lack of spontaneity, decreased interest in daily living activities, or impaired judgment. Some behavioral changes include, but are not limited to, excessive manual exploration of the environment, withdrawal from social interaction, inability to function or interact in social or personal situations, inability to maintain employment, decreased ability to function in self care, or behavior that is inappropriate relative to the environment.

F. Prion Diseases

Prion diseases are degenerative disorders of the nervous system caused by transmissible particles that contain a pathogenic isoform of the prion protein, a normal constituent of cell membranes. Although it is not necessary to understand the mechanism of an invention, it is believed that pathogenic prion protein isoforms fold abnormally and when integrated into the plasma membrane cause other proteins to function improperly.

Prion diseases are primarily represented by spongiform encephalopathies including, but not limited to, Creutzfeldt-Jakob disease, Gerstmann-Straussler-Scheinker syndrome, fatal familial insomnia, Kuru, scrapie, or chronic wasting disease. Some have reported that Spiroplasma species are present in brain tissue recovered from subjects infected with the above diseases, and speculate that this organism may be a causative factor.

The most common human prion disease is Creutzfeldt-Jakob disease (CJD). Most cases are sporadic with an unknown mode of transmission. 10-15% of cases are inherited, while a small number have been transmitted by medical procedures.

The spread of human prion diseases through consumption of infected material has been implicated historically in kuru and recently in variant CJD. Animal prion diseases (scrapie of sheep, transmissible mink encephalopathy, chronic wasting disease of cervids, and bovine spongiform encephalopathy) all seem to be laterally transmitted by contact with infected animals or by consumption of infected feed. The different modes of transmission of different prion diseases, the unpredictable species barriers, the variable distribution of infectivity in tissues, and strain variations found in some diseases all make risk assessment and predictions of future events difficult.

Some symptoms of prion related diseases include, but are not limited to, personality changes, hallucinations, muscle twitching, muscle stiffness, nervous, jumpy feelings, changes in gait (walking, locomotion), lack of coordination—stumbling, falls, speech impairment, poor enunciation (hard-to-understand speech or mumbling), sleepiness, delirium or dementia develops rapidly, deterioration in all aspects of brain function, or profound confusion and/or disorientation.

IV. Electromagnetic Imaging

Free radicals include molecules having one or more unpaired electrons in their outer orbitals. A number of related magnetic resonance (MR) methods are used for imaging free radicals, all of which make use of the fact that a free radical's unpaired electron exhibits a quantum-mechanical spin and therefore has a magnetic moment. An MR signal can be generated in a manner analogous to the detection of hydrogen nuclei (protons) by nuclear magnetic resonance (NMR) in conventional MRI. MR of unpaired electrons is called electron spin resonance (ESR). Substances that have unpaired electrons are termed paramagnetic, thus ESR is also referred to as electron paramagnetic resonance (EPR). The mass of an electron is about three orders of magnitude smaller than that of a proton; therefore the MR properties of the two particles are rather different. In a given strength of applied magnetic field, the ESR frequency is 659 times that of proton NMR. For example, in a magnetic field of 1 T (a common field strength for clinical MRI), the NMR frequency is 42.6 MHz while the ESR frequency would be 28 GHz, well into the microwave part of the electromagnetic spectrum, with strong absorption by conducting samples such as tissue. Owing to this, most biomedical free radical imaging by ESR is carried out at lower magnetic field strengths, preferably between 10 and 40 mT.

Another difference between ESR and NMR is that the electron relaxation times of free radicals are shorter, typically between 0.1 μs and 1 μs, a million times shorter than those encountered in clinical MRI. As a result, most ESR spectroscopy and imaging is done using “continuous wave” (CW) detection methods. Instead of applying a pulse of radiowave energy and waiting for the transient response, as in clinical MRI, in CW ESR the sample is continuously irradiated with low intensity electromagnetic radiation and the resonant response of the unpaired electrons is measured by slowly increasing the strength of the applied magnetic field. When an ESR is encountered, the combined effect of the unpaired electrons (the “electron magnetization”) alters the electrical properties of the resonator used to apply the radiowaves and a reflected signal can be measured. To obtain spatial information about the sample, a magnetic field gradient is applied continuously during the magnetic field sweep. An image is built up by back-projecting a series of one-dimensional projections of the sample, obtained by measuring the ESR signal repeatedly, with the direction of the applied magnetic field gradient stepped in small increments.

Using a longitudinally-detected ESR (LODESR), the sample is irradiated with electromagnetic radiation close to the desired resonant frequency (e.g., 300 MHz), the intensity of which is modulated at a lower frequency (e.g., 0.5 MHz). This causes the unpaired electron magnetization to oscillate at twice the modulation frequency, and the signal is detected by a receiver coil oriented along the same direction as the applied magnetic field.

The technique known as proton-electron double resonance imaging (PEDRI) is based on the Overhauser effect. PEDRI uses a combination of ESR and MRI; the ESR of a free radical of interest is irradiated during the collection of an MR image. The Overhauser effect causes an increase in the NMR signal strength in parts of the sample containing free radicals, and these regions are revealed by an increased intensity in the final image. PEDRI uses standard MRI software and hardware, but requires the additional capability of irradiating the sample at the ESR frequency. Field-cycled PEDRI (FC-PEDRI) allows free radical imaging in humans and works by switching the applied magnetic field between two values during the MRI pulse sequence. A very low magnetic field (i.e., for example, 3 mT) is applied while ESR irradiation is taking place, so that this can be at a low frequency (i.e., for example, 50 MHz) that can easily penetrate the body. The field is then increased to a much a higher value (i.e., for example, 60 mT) so that the NMR signals can be measured with improved signal-to-noise ratio and therefore good image quality. Additional methods are described in the literature. Pursley et al., “Integration of digital signal processing technologies with pulsed electron paramagnetic resonance imaging” J Magn Reson. 2005 Oct. 19 [e-publication, ahead of print]; Lurie & Mader “Monitoring drug delivery processes by EPR and related techniques—principles and applications” Adv Drug Deliv Rev. 2005 Jun. 15; 57(8):1171-90 both herein incorporated by reference.

In some embodiments, the invention relates to MRI monitoring of the mitochondria of dopaminergic neurons using any compound disclosed herein, preferably, TP-TEMPO. Fully functional mitochondria have a large transmembrane potential, which allows them to accumulate lipophilic cationic probes and drugs. Radicals contain an unpaired electron and so have a magnetic moment or spin. This means that they can be detected by a number of magnetic resonance spectroscopic techniques, including MRI. Nitroxides are commonly used in human patients as MRI contrast reagents in many organs and tissues but these techniques do not target either SN dopaminergic neurons themselves or dopaminergic neuronal mitochondria.

It is not intended that contrast agents contemplated by this invention be limited to any particular structure of the chemical compound provided it has a hydropyridine group and a nitroxide group and functions to provide a radical magnetic moment in a dopaminergic neuron upon administration to a subject. In one embodiment, a specific contrast agent for active mitochondria of dopaminergic neurons of the SN comprises P⁺-TEMPO.

Although it is not intended that the present invention be limited by any particular mechanism, it is believed that TP-TEMPO, a water-soluble molecule, is able to cross the blood brain barrier and enter the brain. See FIG. 7. Once in the brain, TP-TEMPO is converted into the P⁺-TEMPO and specifically taken up by dopaminergic neurons. Once within the dopaminergic neurons, P⁺-TEMPO is taken up by a mitochondrial dopamine transporter and accumulates within the mitochondria. Conventional MRI imaging techniques are limited to examining only the general size of the SN and cannot differentiate between neurons and other cell types. TP-TEMPO/P⁺-TEMPO contrast agent imaging can not only provide an estimation of the number of SN dopaminergic neurons, but can also monitor cell viability. Although it is not necessary to understand the mechanism of an invention, it is believed that the uptake of P⁺-TEMPO by SN dopaminergic cells and their respective mitochondria is an energy driven process, and as such, accumulated concentrations is directly proportional to overall cellular energy levels.

Selenium 77 (Selenium-77) is a stable (non-radioactive) isotope of selenium. One can enrich composition comprising selenium isotope 77 by commercially purchasing selenium 77 metal, from e.g., American Elements, and using it to prepare the reagents used herein, e.g., the reaction of selenium metal with a reducing agent such as sodium borohydride in a protic solvent such as ethanol as provided or using procedures appropriately modified as disclosed in Klayman and Griffin, J. Amer. Chem. Soc. 1973, 95, 197.

### EXPERIMENTAL

The following examples are only representative of some embodiments contemplated by the present invention and are not intended to provide any limitations.

### Example 1

### Synthesis of Mn-Salens

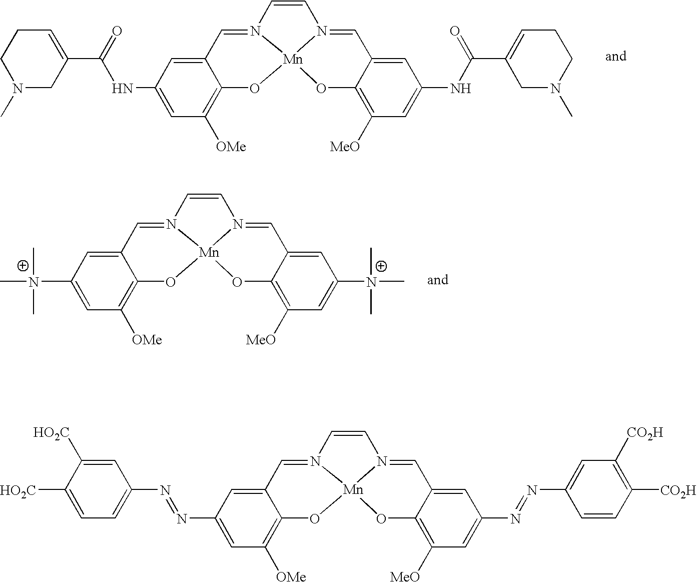

EUK-134 is made from the condensation of o-valinin and ethylene diamine. Similarly MP-EUK 134 is made from the condensation of ethylene diamine and 2-hydroxy-3-methoxy-5-nitrobenaldehyde. Further reduction of the nitrile is accomplished by mixing with Zn powder and 0.1 M HCl to provide the aniline purified by water extraction and alkali precipitation in ethanol. Acecoline hydrobromide is obtained by de-esterification of methyl 1,2,5,6-tetrahydro-1-methylnicotinate in 0.1 M HCl at 65 degrees C., and coupled to the aniline. Reaction of the aniline with excess iodomethane provides the trialkylated product. These products may be converted to the triphenyl or triethyl ammonium manganese salens. Reaction of the aniline with NaNO₂ in ethanol and acetic acid followed by KH-phthalate provides the corresponding azo compound.

### Example 2

### Synthesis of 1,4-diaza-6-oxo-2-selenacyclohexan-4-oxyls

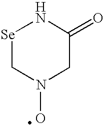

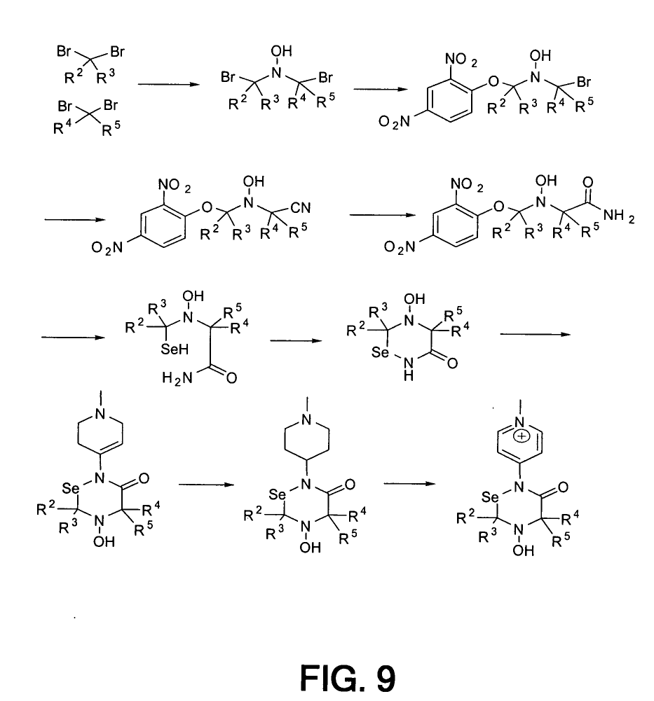

The compound above is prepared according to the scheme depicted in FIG. 9. Hydroxylamine is refluxed in dibromomethane in the presence of triethylamine. The N,N, di-bromomethyl hydroxylamine is insoluble in CH₂Br₂ and is taken up by water. 2,4, dinitrophenol and N,N, di-bromomethyl hydroxylamine (1:2) are refluxed in ethanol. The main product (≧90%), 2,4 dinitro(N-methoxy N-bromomethyl hydroxylamine) benzene is a liquid soluble in CH₂Cl₂. This is converted to the nitrile by refluxing with KCN in methanol. The dark red solid is then converted directly to the amine by being heated with 1 gram of KOH/1 mL of tert-butanol, per gram of nitrile, for 20 minutes and then quenched in aqueous KCl. The solvent is removed and the salty yellow oil was then treated with 33% HBr in acetic acid for 10 minutes and neutralized with KOH. The light yellow liquid bromide was desalted following removal of the aqueous phase by washing with absolute ethanol. It is then added to an ethanolic solution of sodium selenide, Na₂Se, (1:2). The liquid was treated with 10 mM H₂O₂ to oxidize the ligand and cause ring closure. Subsequent treatment with silver oxide generates the nitroxide. The double bond in the heterocyclic ring may be reduced in methanol using hydrogen and Pd on carbon catalyst optionally under elevated temperature and pressure.

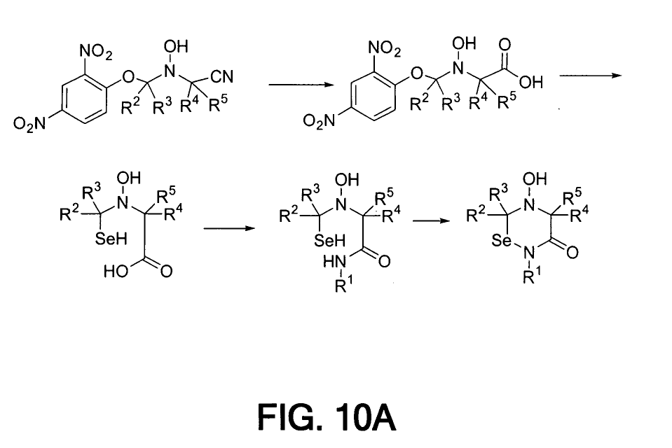

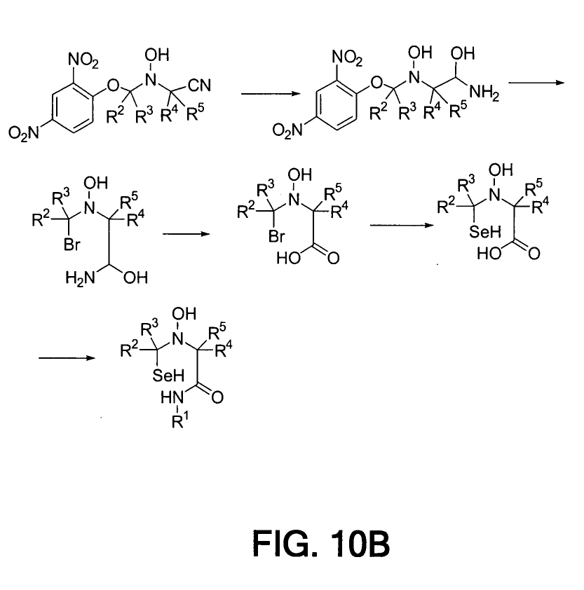

A variety of alternative procedures are contemplated. As depicted in FIG. 10A, the nitrile derivative is oxidized to the carboxylic acid by aqueous sodium hydroxide. Further treatment with hydrogen bromide in acetic anhydride results in a brominated derivative. Any acid bromide is converted to carboxylic acid by water washing. Reaction of the acid bromide or coupling to the carboxylic acid using coupling reagents such as 1-Ethyl-3-(3-dimethylaminopropyl)-carbodiimide (EDC), a water-soluble derivative of carbodiimide. Carbodiimide catalyzes the formation of amide bonds between carboxylic acids or phosphates and amines by activating carboxyl or phosphate to form an O-urea derivative. This derivative reacts readily with nucleophiles such as substituted or unsubstituted amines to provide the desired amide. Formation of the selenol is accomplished by reaction of the brominated derivative with sodium selenide. Subsequent oxidation (e.g., air) is sufficient to provide the cyclized product.

In another alternative, as depicted in FIG. 10B, the nitrile is reduced in the presence of sodium borohydride, exposed to hydrogen bromide in acetic acid and mixed with water to provide the carboxylic acid. Subsequent reaction of the brominated derivative provides the selenol, which is coupled to amines as described above.

Corresponding aromatic produced are obtained by dehydrogenation with palladium, heating in the presence of concentrated sulfuric acid, or mixing with an known or modified aromatase enzyme.

### Example 3

### Synthesis of 1,3-diaza-5-selenacyclohexan-3-oxyls

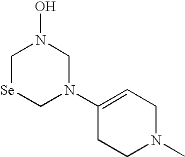

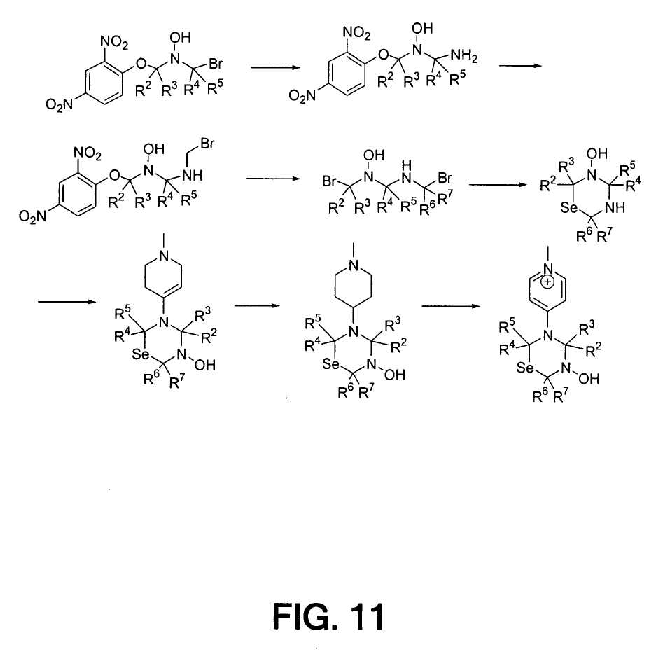

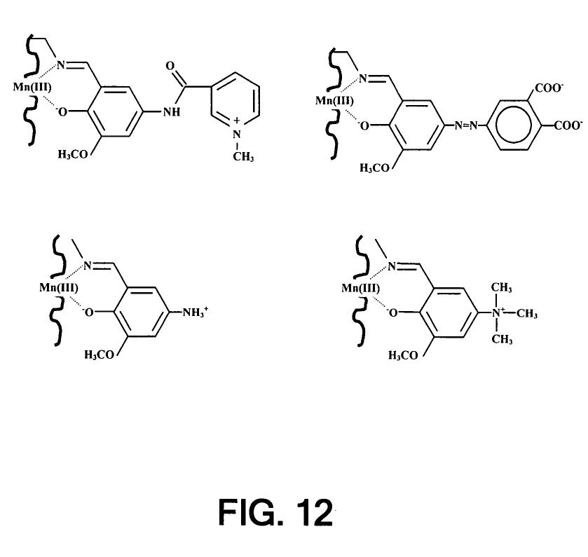

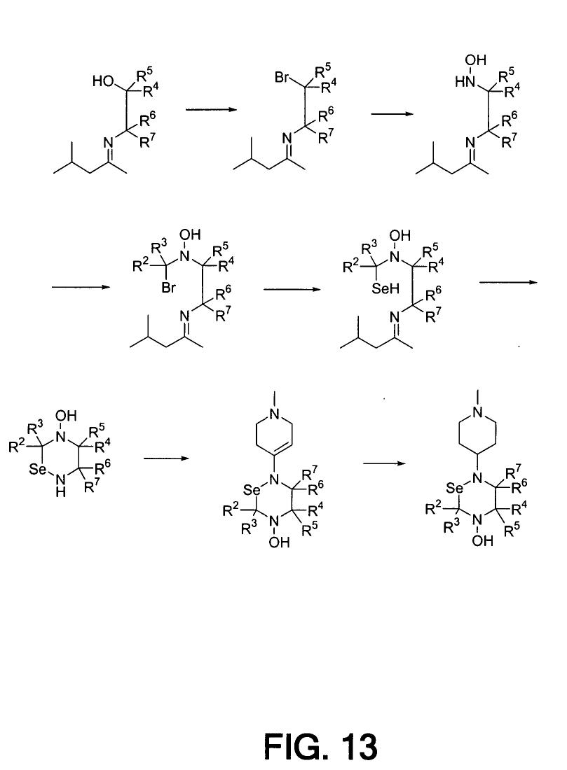

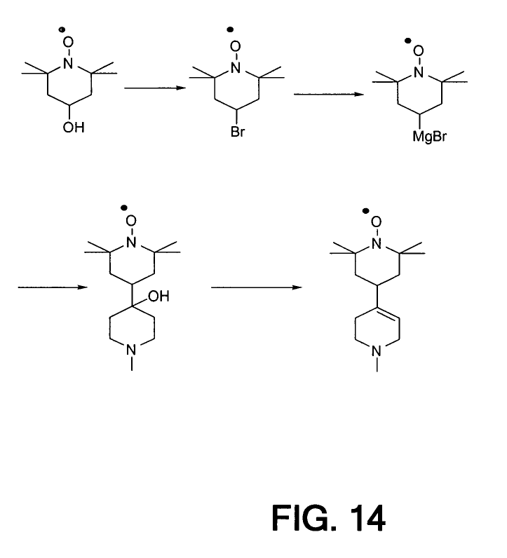

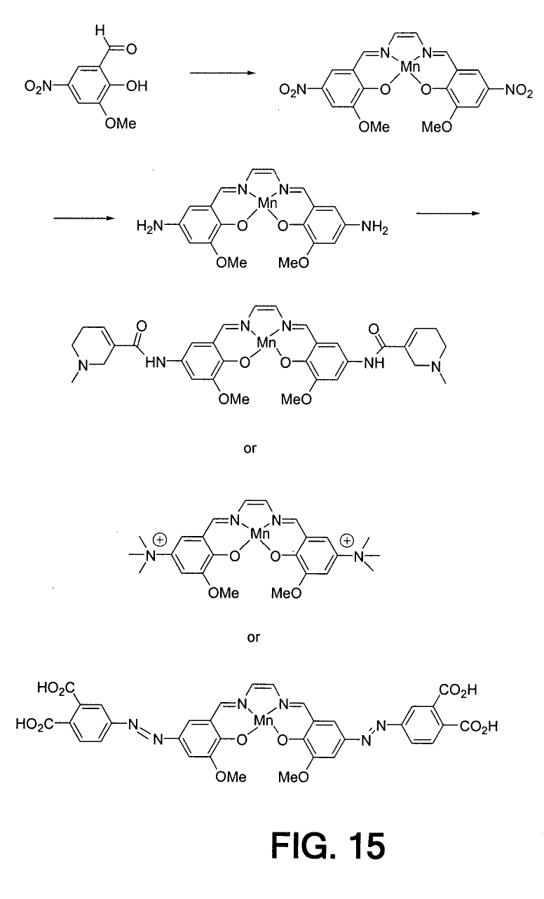

The compound above is prepared according to the scheme depicted in FIG. 11. The brominated derivative is prepared in accordance with Example 2, and as shown in FIG. 9, is reacted by mixing with substituted or unsubstituted amines. This is further reacted with dibromomethane to provide the corresponding brominated derivative. Removal of the protecting group in the presence of hydrogen bromide, acetic anhydride, and water gives the dibrominated derivative that is cyclized in the presence of sodium selenide. The cyclized product is mixed with 1-methylpiperidin-4-one and concentrated sulfuric acid to provide the couple hydropyridine.

### Example 4

### Whole Animal Model for Antioxidant Alleviating Oxidative Stress

One grows nematodes (Caenorhabditis elegans) in the presence of paraquat as disclosed in Keaney et al., (2004) Free Rad. Biol. Med. Vol. 37 239-250. Paraquant catalyses the formation of O₂⁻ from the mitochondrial ubisemiquinone pool. One modulates the paraquat dosage so the lifespan of the animals is almost exactly halved. These animals are systems to study drugs that are capable of alleviating oxidative stress. One observes negation of the toxic effects of paraquat.

### Example 5

### Protection of Neurons from Microglial Inflammatory Stress

One treats co-culture of primary rat neurons and microglia with inflammatory LPS/IFN-gamma/PMA for 24 hours causing activation of the microglia and generating neurotoxic RO/NS. Incubation causes 90% of the neurons to die while exposure to a test compound (e.g., TP-MnSalen/TP-EUK-134) under identical conditions results in less (14% for TP-MnSalen) neuronal death.

### Example 6

### Ischemia/Reperfusion Injury in Kidney and Multiple Organ Failure in Endotoxic Shock

One treats wild-type flies with paraquat sufficient to kill 50% of flies in 48 hr due to oxidative stress and on null mutant Sod2 flies, which exhibit a severely reduced adult life span as disclosed in Duttaroy et al., (2003) Genetics 165, 2295-2299. One evaluates the compounds disclosed herein with mutant fly lines that show defects in mitochondrial biogenesis and reduced life span as a result of mutations in mitochondrial DNA (mtDNA) replication genes including the replicative DNA helicase, mitochondrial single-stranded DNA-binding protein and the mitochondrial replicase, DNA polymerase gamma.

### Example 7

### Permanent Middle Cerebral Occlusion (MCAO) Mouse Model of Stroke

One evaluates the effects of compounds disclosed herein on a mouse MCAO model by comparing infarct size, markers of RO/NS, and apoptosis markers with both control animals (i.e., no MCAO and no neuroprotective treatment) and typical infarcts achieved with MCAO alone and no neuroprotective treatment. Infarcts generally occur on the hemisphere of the brain ipsilateral to the occluded vessel and encompass an approximate region supplied by the middle cerebral artery. One stains sections with triphenyltetrazolium chloride (TTC), which stains viable cells a pink color. White unstained regions represent dead tissue.

### Example 8

### Ischemia/Reperfusion Injury in the Kidney

When compared to organs obtained from sham-operated rats, the kidneys and the liver of rats subjected to endotoxemia show substantial histological alterations consistent with shock-induced organ injury. One reduces the degree of organ injury (as assessed by histology) caused by endotoxemia in kidneys and the liver of rats, then when one treats with a test compound using methods disclosed in Chatterjee et al, (2004) Am J Nephrol. 24, 165-77 and D'Emmanuele di Villa et al., (2003) Euro. J. Pharmacol. 466, 181-189. The kidneys and liver of rats subjected to endotoxemia exhibit a marked staining for nitrotyrosine when compared to organs obtained from sham-operated rats. In contrast, one reduces the degree of nitrotyrosine staining in these organs in rats one pretreats with the test compound.

### Example 9

### Escherichia coli Lipopolysaccharide Endotoxic Model of Shock

One injects rats with a lipopolysaccharide from the bacterium, E. coli, providing an overly aggressive immune response. Immune cell generate NO causing nitrosative stress in all the major organs, leading eventually to multi-organ failure. In humans this type of pathology is directly comparable to toxic shock syndrome. One administrates a test compound preventing multi-organ failure.

### Example 10

### Caspase-3 Assay

One transfers human neuroblastoma cells (SH-SY5Y) to 75-cm² filter vent flasks, grown in Dulbecco's modified Eagle's medium containing 10% fetal bovine serum (FBS), L-glutamine (4 mmol/liter), penicillin (100 units/mL), and streptomycin (100 μg/mL) and incubates at 37° C. in a humidified atmosphere of 5% CO₂ and 95% air. One seeds cells in six-well dishes and grown to a confluence of 70-80%. Twelve hours before the start of treatment, one replaces the medium with Dulbecco's modified Eagle's medium containing 2% FBS. One incubates neuroblastoma cells with 150 μM H₂O₂ and then by 25 μM of test compound for 6 hours. Following the termination of incubation, one collects the cells by gentle scraping, washing three times with DPBS, and suspending in 100 μl of lysis buffer and passing through a 24-gauge needle 10 times to ensure complete lysis. One centrifuges the lysate is at 4° C. for 10 min at 10,000 rpm. One uses 50 μl of the clear supernatant for the activity assay according to the manufacturer's protocol. The increase in the optical density at the appropriate wavelength is an index of caspase-3 activity.

Caspase-3 (CPP32/apopain), which has a substrate specificity for the amino acid sequence Asp-Glu-Val-Asp (DEVD) and cleaves a number of different proteins, including poly(ADP-ribose) polymerase (PARP), DNA-dependent protein kinase, protein kinase Cδ and actin, has been shown to play a role in initiation of apoptosis. The EnzChek® (Invitrogen) Caspase-3 Assay Kit #1 allows the detection of apoptosis by assaying for increases in Caspase-3 and other DEVD-specific protease activities (e.g., Caspase-7).

The basis for the assay is that 7-amino-4-methylcoumarin-derived substrate Z-DEVD-AMC (where Z represents a benzyloxycarbonyl group), which is weakly fluorescent in the UV range (excitation/emission ˜330/390 nm), yields a bright blue-fluorescent product (excitation/emission ˜342/441 nm) upon proteolytic cleavage. EnzChek® can be used to continuously monitor the activity of Caspase-3 and other closely related proteases in cell extracts and purified enzyme preparations using a fluorescence microplate reader or fluorometer. In addition to the Z-DEVD-AMC substrate, the EnzChek® Caspase Assay Kit #1 contains the reversible aldehyde inhibitor Ac-DEVD-CHO, as well as the reference standard 7-amino-4-methylcoumarin (AMC). The Ac-DEVD-CHO inhibitor can be used to confirm that the observed fluorescence signal in both induced and control cell populations is due to the activity of caspase-3-like proteases. The reference standard is included to allow quantitation of the amount of AMC released in the reaction.

### Example 11

### Superoxide Dismutase Assay

One mixes dimethyl sulfoxide solution of potassium superoxide (2 mM) with a buffer solution of a putative SOD mimic in a stopped-flow spectrometer system. One dilutes the superoxide solution at least 17-fold after mixing and to give an initial concentration of superoxide between approximately 60-120 μM. The initial concentration of superoxide is kept at least 10 times above that of the concentration of the compound. Consequently, one tests the compounds for SOD activity in the concentration range of 5×10⁻⁷ M to 6×10⁻⁶ M. The buffer solution is 60 mM HEPES, pH 8.1. One monitors the decay of superoxide spectrophotometrically at 245 nm, the absorbance maximum for superoxide. One processes the absorbance data using personal computer to obtain an observed rate constant (k_{obs}) from the slope of a plot of ln(A^{∞}−Aₜ) versus time. One determines the catalytic rate constant (k_{cat}) from the slope of a plot of k_{obs} versus catalyst concentration. One establishes a compound to be inactive as an SOD catalyst when the decay of superoxide is unperturbed self-dismutation rate obtained in the absence of compound.

### Example 12

### Synthesis of 1,4-diaza-2-selenacyclohexan-4-oxyls

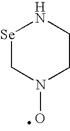

Ethanolamine (50 mL), was mixed with 100 mL of 4-methyl-2-pentanone and refluxed at 82-85 degrees. This forms two phases with the ethanolamine on the bottom. Approximately 30 mL of H₂O: 4-methyl-2-pentanone was distilled off, and the vessel was recharged with 125 mL of 4-methyl-2-pentanone. By this time there was a single-phase system that had become a pale orange imine.

Acetone, 100 mL, and 8 mL of PBr₃ are added to 36 grams of imine. The reaction is very exothermic and done over the course of tens of minutes, after 4 mLs was added, a change in the solution was observed with the formation of an orange wax. After 10 minutes of stirring, NaHCO₃ was added along with 100 mL of ethanol. The solution was filtered twice to remove the solids. Ethanol and acetone was removed from the solution by vacuum distillation. Excess hydroxylamine was added, heated for 30 min. To the solution was added 30 mL CH₂Br₂. The resulting solution was refluxed and distilled of solvent at 70 degrees C. under reduced pressure, cooled under a cold-water tap and washed with 2×100 mL of water and chloroform. The bottom chloroform layer was separated from the water layer. A little 4-methyl-2-pentanone was added and the chloroform solution was distilled off to provide the brominated imine.

Sodium borohydride (NaBH₄) was added to a solution of selenium (4 g) in 50 mL ethanol until the solution is clear. The solution is transferred to the brominated imine. The solution becomes bright red. The ethanol is removed by distillation under vacuum. Adding more NaBH₄ gets rid of red color. The solution is then heated. When ethanol levels drop and a dark red solution forms, additional water and a little NaBH₄ was added to give a yellow solution. The solution is allowed to stir in order to hydrolyze the imine and promote ring formation. Solvents are removed to isolate the desired product.

### Example 13

### Synthesis of 2,2,6,6-tetramethyl-4-(1-methyl-1,2,3,6-tetrahydropyridin-4-yl)piperidin-1-oxyl (TP-TEMPO)

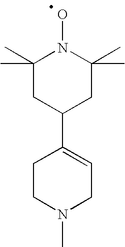

4-Hydroxy-TEMPO was brominated in the presence of PBr₃ and methylene chloride. The resulting 4-bromo-TEMPO was turned into the corresponding Mg-Grignard reagent and condensation with N-methyl-4-piperidone provided the desired tertiary alcohol. Subsequent dehydration and purification gives 2,2,6,6-tetramethyl-4-(1-methyl-1,2,3,6-tetrahydropyridin-4-yl)piperidin-1-oxyl.

4-Hydroxy-TEMPO and triethylamine is dissolved in dichloromethane and chilled in ice water. Methansulfonic acid chloride is added the solution and stirred for three hours then washed with water and 5% NaHCO3. The compound was placed in a flask with sodium sulfate and recrystallized in chloroform/hexane solution. In acetone, the compound is refluxed with lithium bromide for 30 minutes. After cooling the product is extracted into ether and dried. The product refluxed for 1 hour with Mg in dry tetrahydrofuran. The solution is alkalinized with CaCO₃ and extracted in chloroform. The solvent is removed. The product is taken up in ethanol, acidified with HCl, and filtered.

### Example 14

### Synthesis of 4,5-dimethyl-2-(1-methyl-1,2,3,6-tetrahydropyridin-4-yl)-1,2-selenazol-3(2 h)-one and 4,4,5,5-tetramethyl-2-(1-methyl-1,2,3,6-tetrahydropyridin-4-yl)-1,2-selenazolidin-3-one

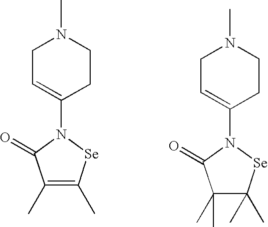

4,5-dimethyl-1,2-selenazol-3(2H)-one or 4,4,5,5-tetramethyl-1,2-selenazolidin-3-one is heated in N-methyl-4-piperidone with concentrated sulfuric acid to provide the corresponding product.

### Example 15

### Synthesis of 5-hydroxy-4,4,6,6-tetramethyl-1,2,5-selenadiazinan-3-one

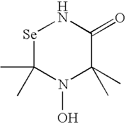

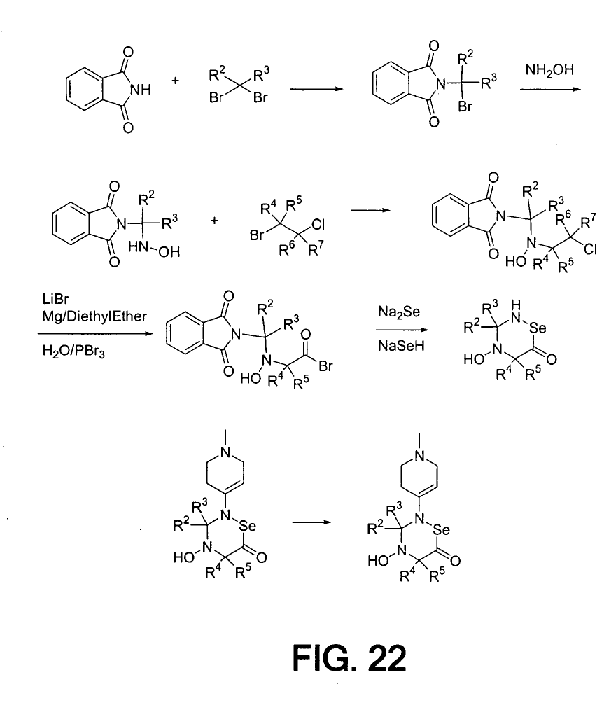

FIG. 22 outlines the synthesis of this embodiment. The applicant found that using appropriately modified procedures (a phthalimide-based Gabriel synthesis) was better than using dinitropyridine intermediates as exemplified in example 2.

### Example 16

### Synthesis of 2,2,4,4,6,6-hexamethyl-1,3,5-selenadiazinane-3,5-diol

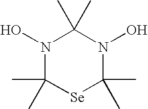

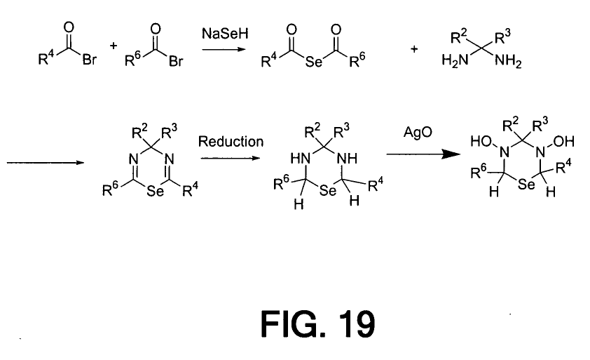

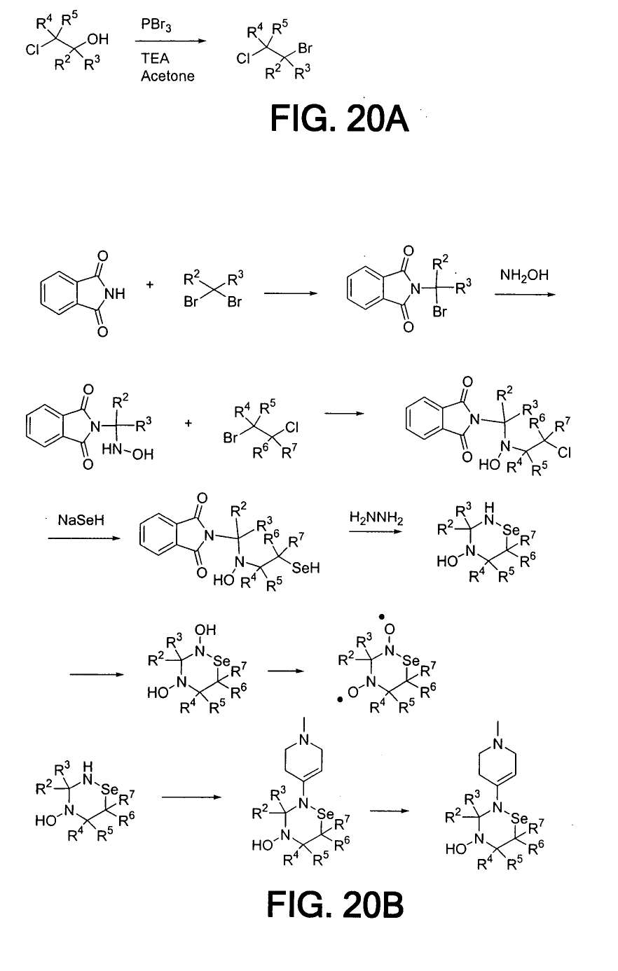

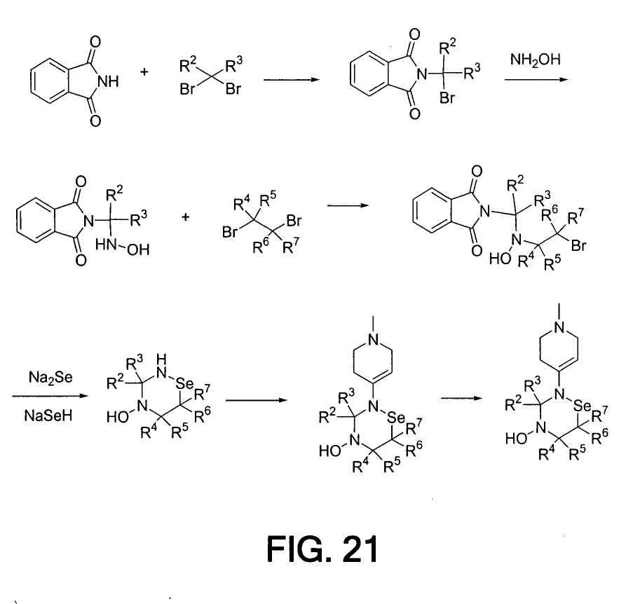

FIGS. 19 and 20 outline the synthesis of this embodiment. The condensation of hydroxylamine with organobromides is preferred with usually >95% yield to give either —C—N(OH)—C— or HN(OH)—C—N(OH)H, depending on whether the hydroxylamine or dibromide is in excess. NaSeH and Br—C(XY)—Br gives HSe—C(XY)—SeH or Br—C(XY)—Se—C(XY)—Br again depending whether NaSeH or Br—C(XY)—Br is in excess. During the course of the synthesis it was discovered that hydroxylamine will generally react with C—Br, but not C—Cl, but NaSeH reacts with both.

### Example 17

### Synthesis of 3,3,5,5-tetramethylselenomorpholin-4-ol

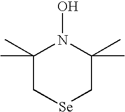

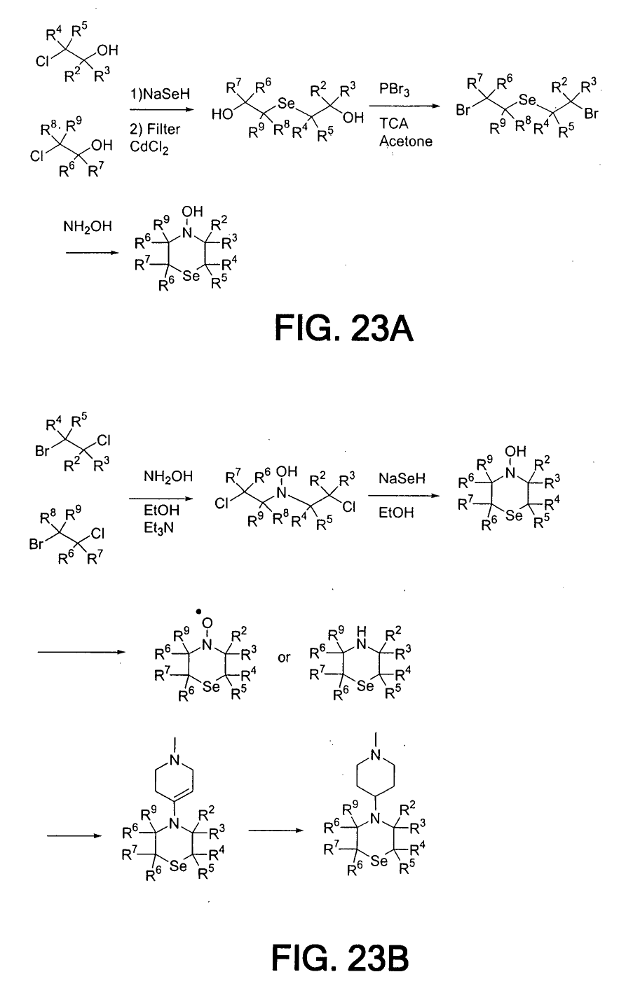

FIGS. 23A and 23B outline the synthesis of this embodiment. During the course of synthesis it was discovered that hydroxylamine will generally react with C—Br, but not C—Cl, but NaSeH reacts with both. Thus, the applicant used Cl—C—C—Br and first added NH₂OH to get Cl—C—C—N(OH)—C—C—Cl, and then added NaSeH to get a complete ring, all in one pot. The NOH/Se rings tend to be drawn to chloroform. Alternatively, adding Br—C—Se—C—Br to H—N(OH)—C—N(OH)N—H may be done.

### Example 18

### Synthesis of 3,3,5,5-tetramethyl-1,2,4-selenadiazolidin-4-ol

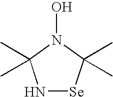

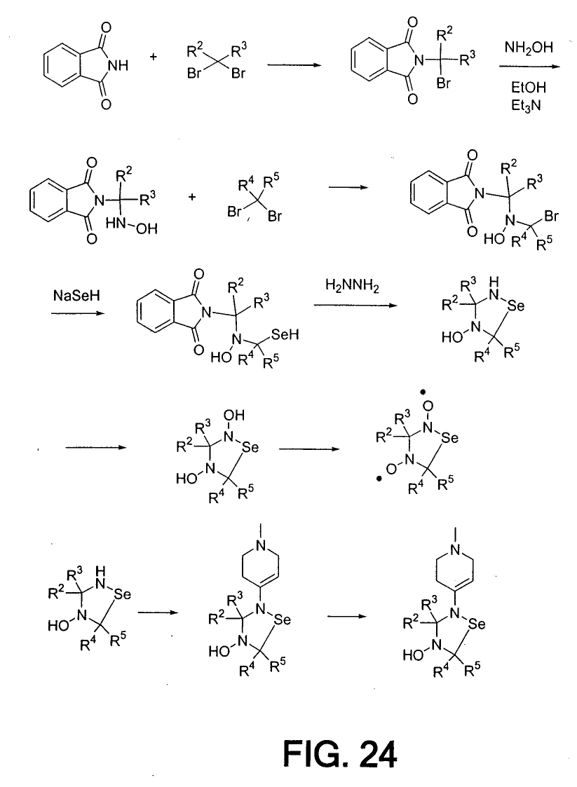

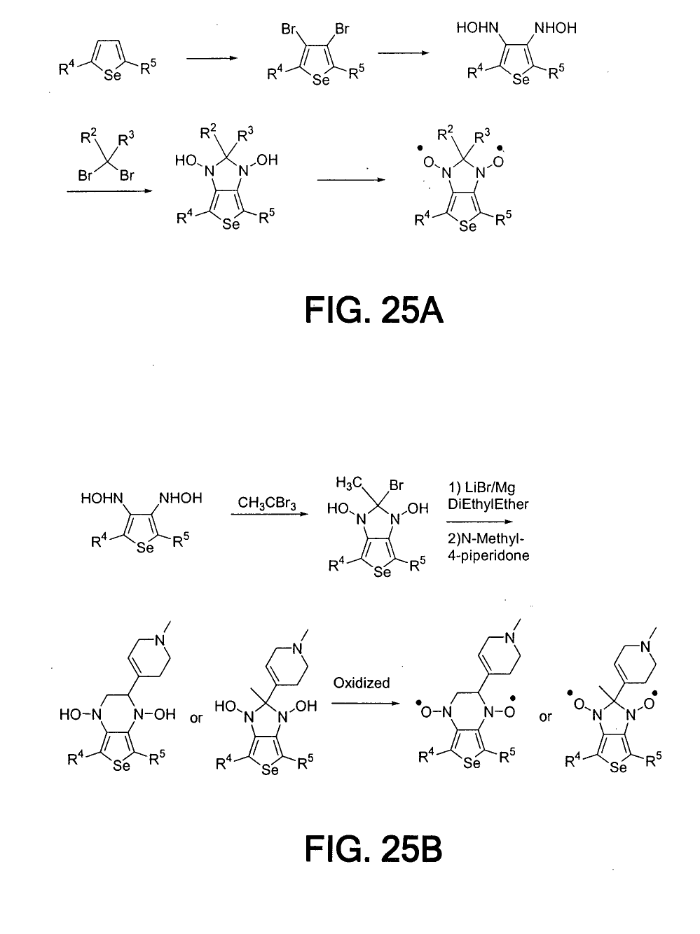

FIG. 24 outlines the synthesis of this embodiment and it was accomplished by using appropriately modified procedures as disclosed in Example 2.

### Example 19

### Synthesis of Ebselen

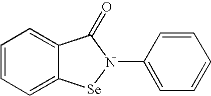

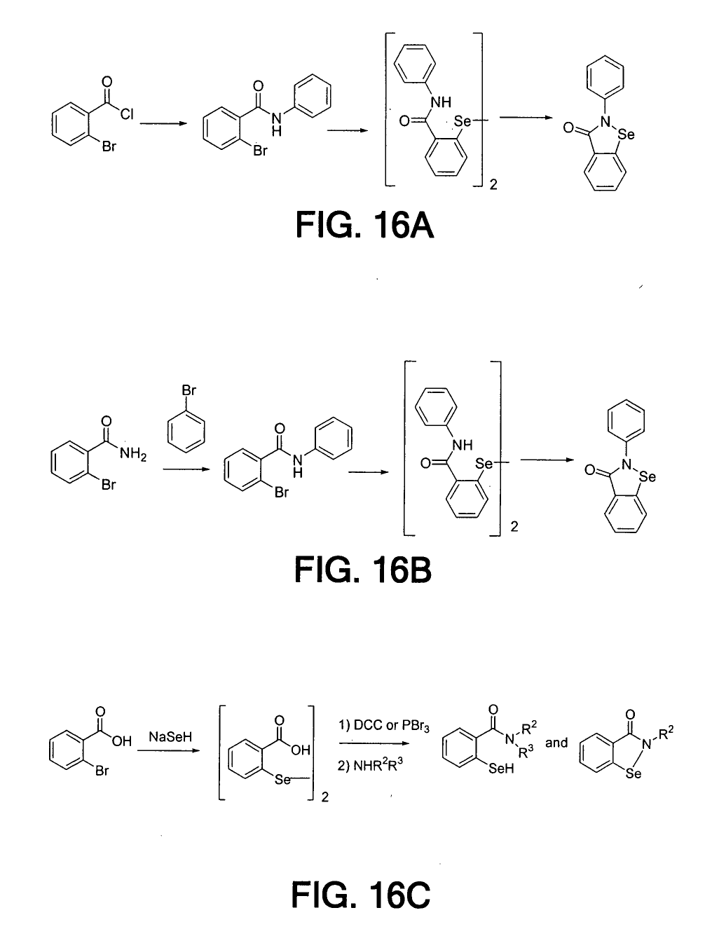

The synthesis is outlined in FIG. 16. To 30 ml of acetonitrile one adds 6 ml of aniline (6.13 grams) and then adds 6.5 ml of bromo-benzoylchloride (10.91 grams) to a flask. One adds water/KOH (100 mM KOH final), filters off white precipitate and dries (96%). One places 4 grams, 14.5 mM, of the intermediate (MW 276.13), adds 1.2 grams of selenium, 12.6 mM, and approx 1.2 gram of NaBH₄ in 100 ml of ethanol, and stirs for an hour. After one hour, one filters off solids and washes with ethanol. One evaporates of the ethanol off under reduced pressure. One adds 2.0 grams of DTT (dithiothreitol, 15 mM) in 5 mL of water. One evaporates the solution to about 30 mL and one adds 200 mL of cold water. One vacuum filtrates the precipitate, and further washes with 200 mL of cold water. One dissolves the precipitate in chloroform and again washes with water. After combining organic layers, one distills off chloroform and, to provide the product, Ebselen, one crystallizes Ebselen in 2-butanone.

### Example 20

### Synthesis of 2-(1-methyl-1,2,3,6-tetrahydropyridin-4-yl)-1,2-benziso selenazol-3(2H)-one

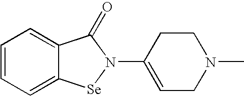

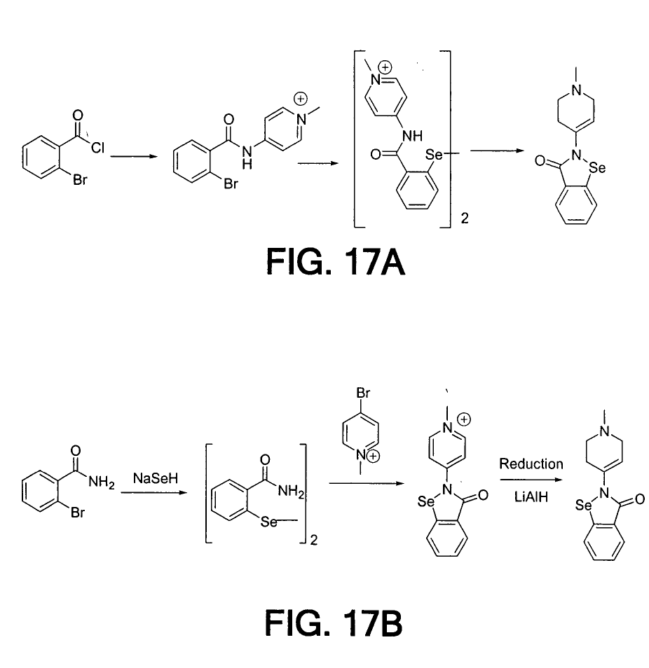

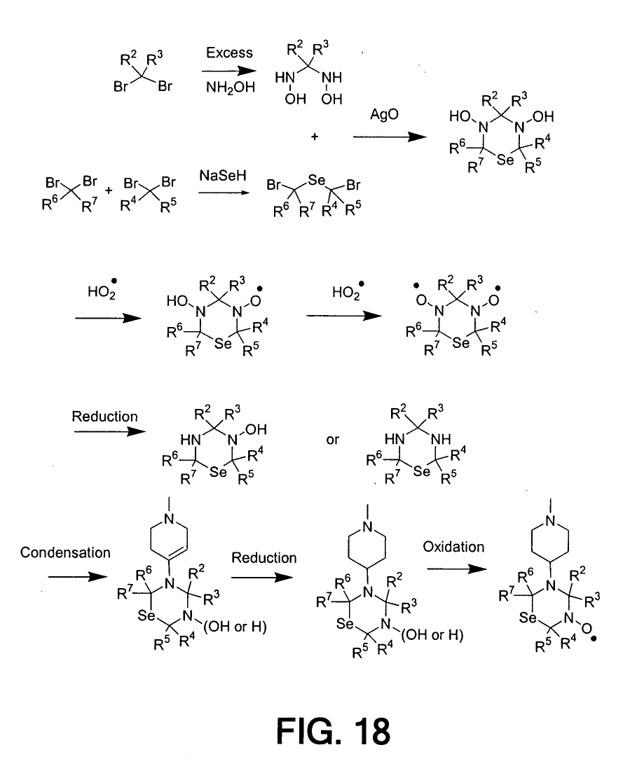

FIG. 17 outlines the synthesis of this embodiment. Using the procedures in Example 19, one makes the N-methylpyridine derivative, which is reduced to a N-methyl tetrahydropyridine derivative. One substitutes selenium for the aryl bromide using Na₂Se₂ to form the dimer as provided appropriately modified in Zade et al., Angew. Chem. 2004, 116, 4613-4615.

### Example 21

### Magnetic Resonance Imaging (MRI)

In nuclear magnetic resonance imaging one looks at nuclear spins. The applicant does not intend embodiments of the invention to be limited to any particular mechanism, but it is believed that by introducing a compound with an electron spin (ESR), the character of nearby nuclear spins are changed. Therefore, one can use a contrast agent, which has an intrinsic electron spin as a way to pertubate the NMR character of nearby nuclear spins. In practical terms these will generally be water molecules, which will not behave normally when next to a nitroxide.

In certain embodiments, one uses nuclear magnetic resonance measurements to look at the effect of an electron spin-containing reagent. However, in alternative embodiments, one can use a human-sized or other subject-sized ESR scanner.

With its dependence on the more biologically variable parameters of proton density, longitudinal relaxation time (T1), and transverse relaxation time (T2), variable image contrast can be achieved by using different pulse sequences and by changing the imaging parameters. Signal intensities on T1, T2, and proton density-weighted images relate to specific tissue characteristics. Moreover, with MRI's multi-planar capability, the imaging plane can be optimized for the anatomic area being studied, and the relationship of lesions to eloquent areas of the brain can be defined more accurately. Since T2-weighted images are most sensitive for detecting brain pathology, patients with suspected intracranial disease are typically screened using the T2-weighted regime. However, it has proven difficult to apply MRI to Parkinson's disease, as the dompaminergic neurons have the same MRI characteristics as the surrounding tissues, so that the Parkinson's lesions are invisible using conventional MRI and can only be detected using histology post-mortem. Brain slices taken by T2 weighted MRI show very little structural information compared with the histological sections of the same mouse brain, particularly in the area of the SN where the vulnerable dopaminergic neurons are located. Lesions in this region are currently undetectable by conventional MRI techniques.

One can detect nitroxides non-invasively using both electron paramagnetic and nuclear magnetic resonance techniques appropriately modified as disclosed in Kawada et al., (2007) “Use of multi-coil parallel-gap resonators for co-registration EPR/NMR imaging” J. MAG. RESONANCE, 184, 29-38. We have examined the ability of TP-TEMPO to affect the T1 and T2 relaxation rates of water-protons in saline solutions. The relaxation times of aqueous TP-TEMPO solutions in saline were measured in the pharmacological concentration range on a Bruker AM400 instrument in a field of 0.5 T using T1 (TR=800 ms, TE=10.0 ms) and 72 (TR=3000 ms, TE=4.2 ms) spin relation regimes. We have been able to show that TP-TEMPO was detectible using convention MRI.

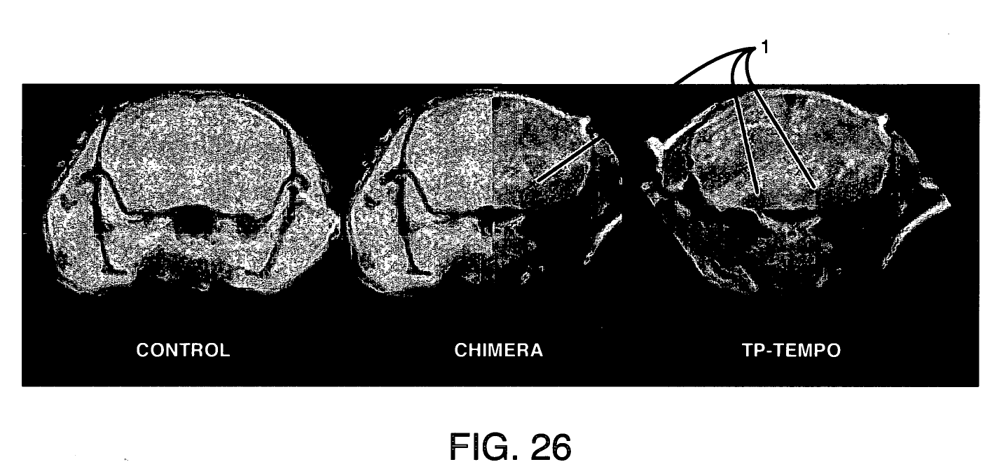

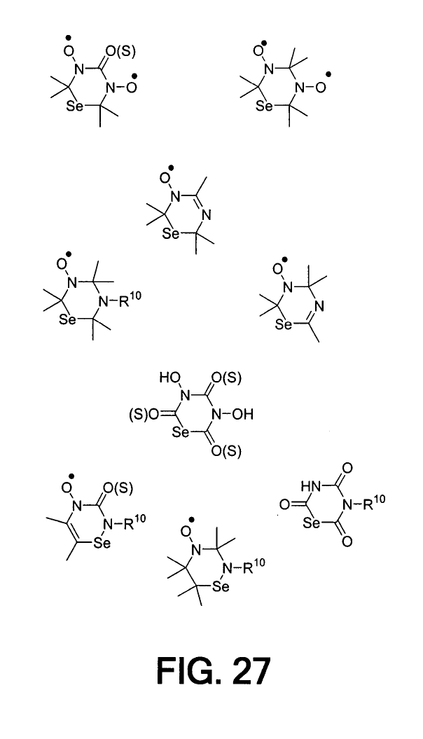

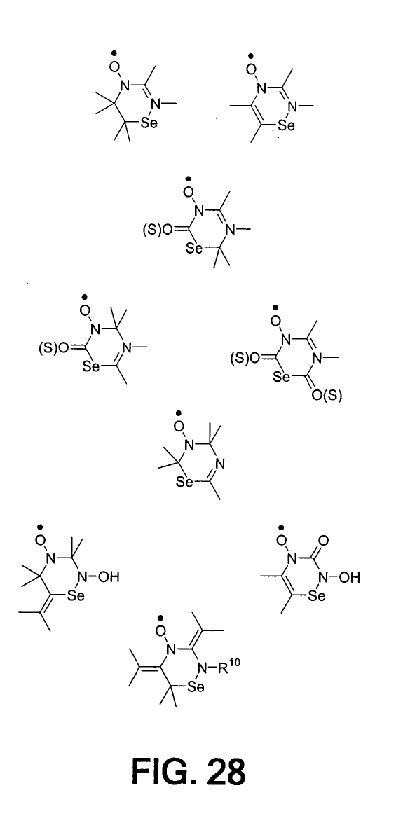

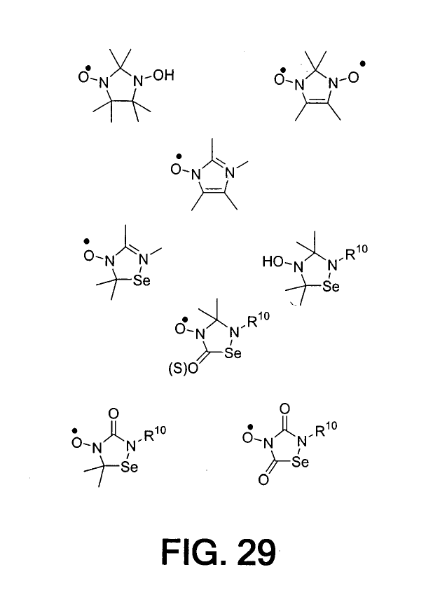

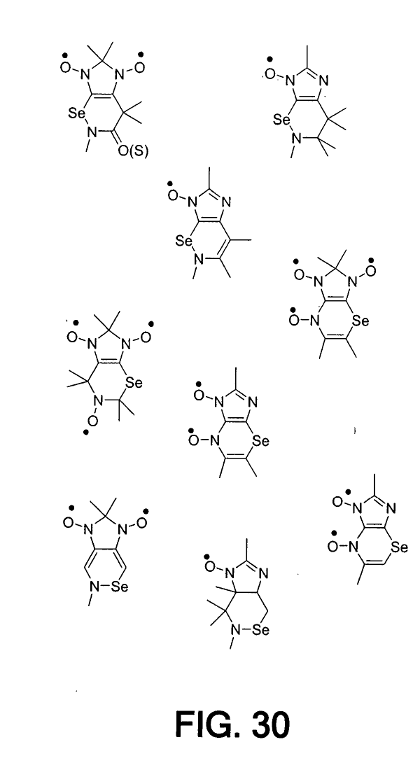

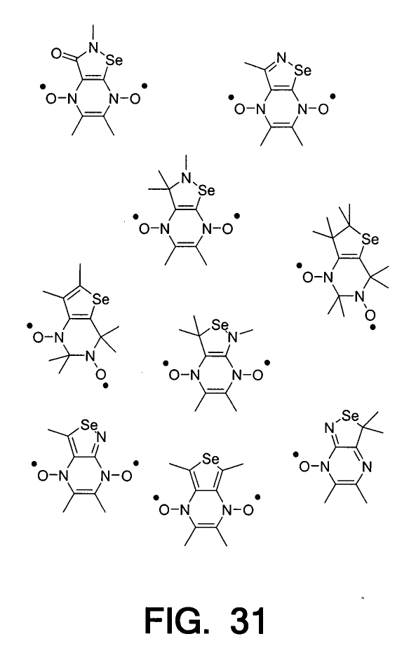

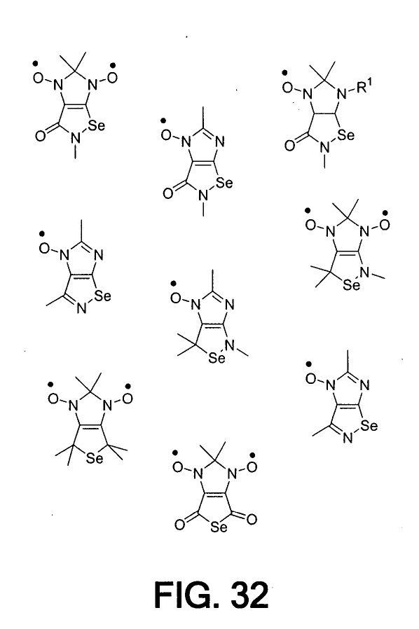

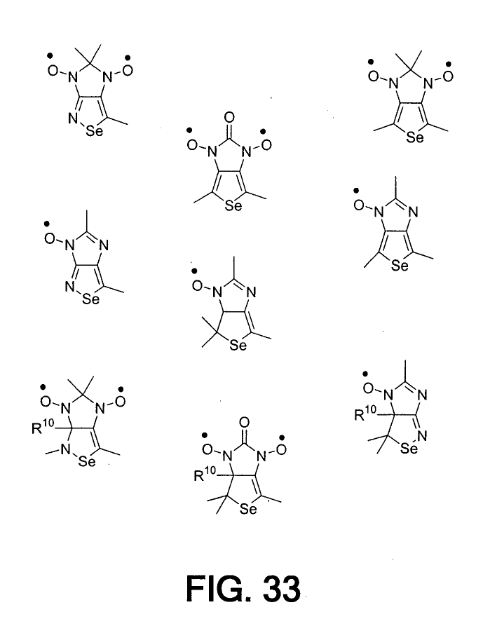

We took images of two mice at 24, 48 and 72 hours after injection with TP-TEMPO. The MRI images taken at 24 hours were unable to show any proton-water MRI signals, with the brain either being totally black or very dark indeed, presumably due to an excess of spin probe. At 72 hours the images generated were not superior to the controls, indicating that the probe has either been excreted from or metabolized by the brain. The optimal signal to noise ratio was found 48 hours after administration. In these images we were able to observe that our probe had not only partitioned preferentially into the brain, but that it had also preferentially migrated to the dopaminergic neurons. We believe that the paradigm postulated above for the bioconversion of TP-TEMPO into P⁺-TEMPO has been demonstrated. In FIG. 26, we show T2 MRI images of taken of two mouse heads, one a control and the other an animal that was treated 48 hours earlier with TP-TEMPO.

We claim:

1. A compound having the following formula: 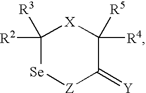
or salts thereof wherein,
Y is ═O, ═S, ═NH, ═N—R⁶, ═N—OR⁶, ═N—SR⁶, ═N—NR⁶R⁷ or two hydrogens each independently bonded to the carbon by a single bond; R², R³, R⁴, R⁵, R⁶, and R⁷ are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl;
X and Z are the same or different and, at each occurrence, independently is N—R¹, N—O°, N—OH, or N═O; and
R¹ is hydrogen,

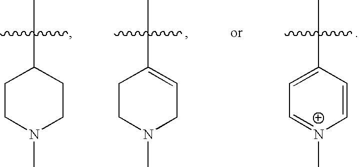

2. The compound of claim 1 wherein said formula is: 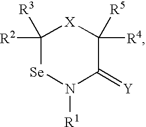
wherein,
Y is ═O or two hydrogens each independently bonded to the carbon by a single bond,
R¹ is hydrogen, 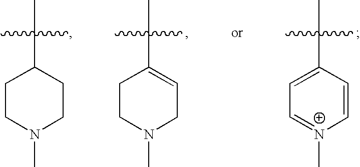
R², R³, R⁴, and R⁵ are the same or different and, at each occurrence, independently hydrogen or alkyl; and
X is N—O°, N—OH, or N═O.

3. A compound having the following formula: 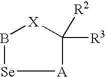
or salts thereof wherein,
A is —CR⁴R⁵—, -Z-CR⁶R⁷—, —CR⁴R⁵—CR⁶R⁷—, or -Z-CR⁴R⁵—CR⁶R⁷—;
B is a single bond, —CR⁴R⁵—, or —CR⁶R⁷—CR⁸R⁹—;
R², R³, R⁴, R⁵, R⁶, R⁷ R⁸, and R⁹ are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl;
X and Z are the same or different and, at each occurrence, independently N—R¹, N—O°, N—OH, or N═O; and
R¹ is hydrogen,

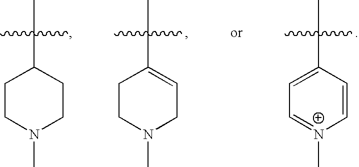

4. The compound of claim 3, wherein said formula is: 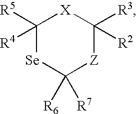
or salts thereof wherein,
R², R³, R⁴, R⁵, R⁶, and R⁷ are the same or different and, at each occurrence, independently hydrogen, alkyl, or substituted alkyl;
X and Z are the same or different and, at each occurrence, independently N—R¹, N—O°, N—OH, or N═O; and
R¹ is hydrogen,

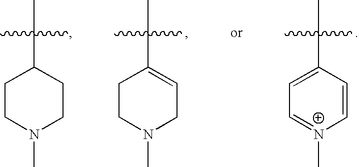

5. The compound of claim 3, wherein said formula is: 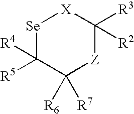
or salts thereof wherein, R
R², R³, R⁴, R⁵, R⁶, R⁷ are the same or different and, at each occurrence, independently hydrogen, alkyl, substituted alkyl;
X and Z are the same or different and, at each occurrence, independently N—R¹, N—O°, N—OH, or N═O; and
and R¹ is hydrogen,

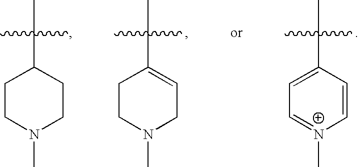

6. The compound of claim 3, wherein said formula is: 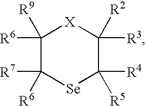
or salts thereof wherein,
R², R³, R⁴, R⁵, R⁶, R⁷ R⁸, and R⁹ are the same or different and, at each occurrence, independently hydrogen, alkyl, substituted alkyl;
X is N—R¹, N—O°, N—OH, or N═O; and
R¹ is hydrogen,

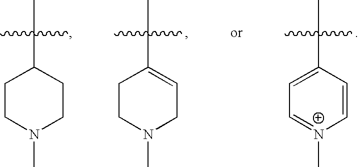

7. The compound of claim 3, wherein said formula is: 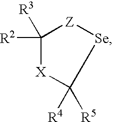
or salts thereof wherein,
R², R³, R⁴, R⁵ are the same or different and, at each occurrence, independently hydrogen, alkyl, substituted alkyl;
X and Z are the same or different and, at each occurrence, independently N—R¹, N—O°, N—OH, or N═O; and
R¹ is hydrogen,

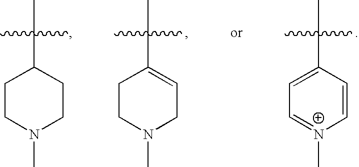

8. A compound having the following formula: 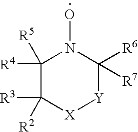
and salts thereof wherein,
X and Y are the same or different and, at each occurrence, independently —CR⁸R⁹—, CH—R¹, or N—R¹, provided that at least one of the group X and Y is N—R¹;
R², R³, R⁴, R⁵, R⁶, R⁷ R⁸, and R⁹ are the same or different and, at each occurrence, independently hydrogen, alkyl, substituted alkyl, aryl, substituted aryl, arylalkyl, substituted arylalkyl, heterocycle, substituted heterocycle, heterocyclealkyl, or substituted heterocyclealkyl; and
R¹ is hydrogen,

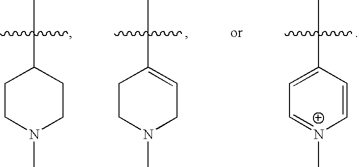

9. The compound of claim 8, wherein
X is CH—R¹;
Y is —CR⁸R⁹—, and
R², R³, R⁴, R⁵, R⁶, R⁷ R⁸, and R⁹ are the same or different and, at each occurrence, independently hydrogen, alkyl, or substituted alkyl.
